(19) United States
(12) Patent Application Publication
(10) Pub. No.: US 2005/0255042 A1
(43) Pub. Date: Nov. 17, 2005

(54) On-demand cleavable linkers for radioconjugates for cancer imaging and therapy

(75) Inventors: Kit S. Lam, Davis, CA (US); Pappanaicken Kumaresan, Sacramento, CA (US); Sally DeNardo, Sacramento, CA (US); Gerald DeNardo, Sacramento, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California Office of Technology Transfer, University of California, Oakland, CA (US)

(21) Appl. No.: 10/997,759

(22) Filed: Nov. 23, 2004

Related U.S. Application Data
(60) Provisional application No. 60/525,236, filed on Nov. 24, 2003.

(51) Int. Cl. A61K 51/00

(52) U.S. Cl. 424/1.49

(57) ABSTRACT

The present invention provides compositions comprising a biological agent, a targeting moiety, and a peptide linker attaching the biological agent to the targeting moiety, wherein the peptide linker is selectively cleaved by a protease. Efficient methods are provided for administering the compositions of the present invention for treating cancer or imaging a tumor, organ, or tissue in a subject. Kits are also provided for administering the compositions of the present invention for radiotherapy or radioimaging.

### CROSS-REFERENCES TO RELATED APPLICATIONS

The present application claims priority to U.S. Provisional Application No. 60/525,236, filed Nov. 24, 2003, which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with support from the U.S. Government under Grant (or Contract) No. P01 CA047829, awarded by the NCI/NIH. The Government has certain rights in this invention.

### BACKGROUND OF THE INVENTION

The dose limiting toxicities of radioimmunotherapy (RIT) are often due to excessive radiation to normal tissues and organs such as liver, kidney, and bone marrow. As a result, new strategies are required to enhance the therapeutic index of cancer-targeting agents by not only improving the delivery of the radiopharmaceutical to the tumor, but also by enhancing the rapid removal of the radioactive agent from normal tissues and organs.

Since the invention of the hybridoma technology (Kohler and Milstein, Nature, 256:495 (1975)), much work has been done on the development of tumor-targeting with monoclonal antibodies (MAbs) (Levy and Miller, J. Natl. Cancer Inst. Monographs, 10:61-68 (1990); Lowder et al., Blood, 69:199-210 (1987); Meeker et al., N. England J. Med., 312:1658 (1985); Meeker et al., Blood, 65:1349 (1985); Miller et al., N. England J. Med., 306:516 (1982); DeNardo et al., Intl. J. Biol. Markers, 2:49-53 (1987); DeNardo et al., “Radiolabeled Monoclonal Antibodies.” S.C. Srivastava ed. Plenum, New York, 152:111 -122 (1988); DeNardo et al., J. Nucl. Med., 36:829-836 (1995); DeNardo et al., Cancer, 80:2706-2711 (1997); DeNardo et al., Anticancer Research, 17:1735-1744 (1997); Frankel et al., Cancer Res., 56:926-932 (1996); Kaminski et al., N. England J. Med., 329:459-465 (1993); Kaminski et al., J. Clin. Onc., 14(7):1974-1981 (1996); Lewis et al., Hybridoma, 14:115-120 (1995); Press et al., N. England J. Med., 329:1219-1224 (1993); Press et al., The Lancet, 346:336-340 (1995)). The clinical success and FDA approval of Rituxan® and Zevalin® (anti-CD 20 MAbs against B-cell lymphoma), Herceptin (anti-HER2/neu MAb against breast cancer), and Mylotarg (anti-CD33 MAb against acute myeloid leukemia) in the treatment of human cancers has further validated the cell-surface targeting approach for cancer therapy. Evaluation of biopsy specimens for the presence of CD20 and HER2/neu is now routinely done for non-Hodgkin's lymphoma and breast cancer, respectively. Several MAbs that target cell surface receptors are also presently in clinical trials.

Antibodies are capable of acting as direct anticancer agents or as targeting agents for delivering an anticancer agent to a tumor. For example, unconjugated antibodies bind to and directly inhibit tumor growth by inhibiting the pro-mitogenic function of specific cell surface receptors (e.g., IMC-C225 (cetuximab), a chimeric anti-EGF receptor MAb; Kim et al., Curr Opin Oncol., 13(6): 506-513 (2001)). Other antibodies are conjugated to toxins (e.g., Mylotarg, a humanized anti-CD33 MAb conjugated to calicheamicin; Sievers and Linenberger, Curr Opin Oncol., 13(6): 522-527 (2001)), and the antibodies act as targeting agents to deliver the toxin to cancer cells. Furthermore, many antibodies are directly conjugated to radionuclides (e.g., radiolabeled anti-CD 20 MAbs such as Zevalin® and Bexxar®), and the antibodies act as targeting agents to deliver the radioactive payload onto cancer cells (Gates et al., J. Nucl. Med., 39:1230-1236 (1998); Liu et al., J. Clin. Onc., 16:3270-3278 (1998); Coiffier et al., Blood, 92:1927-1932 (1998); Wiseman et al., Clin. Cancer Res., 5:3281s-3286s (1999); Foon, PPO Updates, 14:1-11 (2000); Vose, Oncology, 15:141-147 (2001)).

Alternatively, antibodies can be indirectly conjugated to radionuclides through the use bifunctional chelating agents to form radioimmunoconjugates (RICs). Bifunctional chelating agents used in radiometal-labeled RICs have a metal chelating group at one end and a reactive functional group, capable of binding to proteins, at the other end. For example, the macrocyclic chelator DOTA binds yttrium-90 (⁹⁰Y) with extraordinary stability. DOTA also binds indium-111 (¹¹¹In) with considerable stability (Deshpande et al., J. Nucl. Med., 31:473-479 (1990); Moi et al., J. Am. Chem. Soc., 110:6266-6267 (1988)). ⁹⁰Y is an attractive radionuclide for RIT because it provides greater tumor retention and more energetic beta emissions for killing cancerous cells when delivered to a tumor than iodine-131 (¹³¹I) (Sharkey et al., Cancer Res., 48:3270-3275 (1988); Halpern et al., Cancer Res., 43:5347-5355 (1983); Pimm et al., Eur. J Nucl. Med., 11:300-304 (1985)). ¹¹¹In is a gamma-emitting radionuclide that, when incorporated into an RIC, can be used to produce an image of the tumor, or the ¹¹¹In-RIC can be mixed with a corresponding ⁹⁰Y-RIC to track the movement and localization of the ⁹⁰Y-RIC.

The bifunctional macrocyclic chelating agent may be conjugated to the antibody through a reagent such as 2-iminothiolane (2IT) to form an RIC. For example, a bifunctional bromoacetamidobenzyl derivative of DOTA (BAD) has been conjugated to antibodies via 2IT (Deshpande et al., J. Nucl. Med., 31:473-479 (1990); Meares et al., Br. J Cancer, 10:21-26 (1990)). However, ⁹⁰Y clearance from the liver of mice given ⁹⁰Y-2IT-BAD RICs has been appreciably slower than that of corresponding directly radioiodinated MAbs (Halpern et al., Cancer Res., 43:5347-5355 (1983); Pimm et al., Eur. J Nucl. Med., 11:300-304 (1985); Larson et al., Nucl. Med. Biol., 15:231-233 (1988)). Although myelotoxicity is often the dose limiting toxicity in RIT, liver toxicity is also a dose-limiting factor for larger RICs (e.g., whole Ab molecule). For smaller RICs such as single chain antibodies, renal toxicity could be the dose-limiting factor.

Although the use of RICs allows for the site-specific delivery of anticancer agents to a tumor, thus improving the therapeutic index of these anticancer agents, there is also a need to develop strategies for enhancing the rapid removal of the radioactivity provided by the RICs from normal tissues and organs. One approach is to use a pre-targeting strategy (Breitz et al, J. Nucl. Med., 41:131-140 (2000); Goodwin et al., J. of Nucl. Med., 29:226-234 (1988); Goodwin et al., Cancer Res., 54:5937-5946 (1994); Pagamelli, Int. J. Cancer, Suppl. 2:121-125 (1988)), where an unlabeled antibody carrying a chelate-binding site is allowed to accumulate in the target, and then a small, rapidly clearing radiopharnaceutical is administered. The radiopharmaceutical either binds to the pre-targeted site or clears from the body quickly, providing a high tumor-to-background ratio. However, the use of extracorporeal immunoadsorption to remove the free circulating RICs after adequate tumor uptake has been attempted both pre-clinically and clinically, but with only modest success (Dienhart et al., Antibody Immunoconjugates and Radiopharmaceuticals, 7:225-231 (1994); Garkavij et al., J. of Nucl. Med., 38:895-901 (1997); Tennvall et al., Cancer Suppl., 80:2411-2418 (1997); Garkavij et al., Clin. Cancer Res., 5:3059s-3064s (1999)). A recent advance is the use of small antigen-binding fragments of antibodies (Fabs), produced by genetic engineering, to carry radionuclides.

Furthermore, the use of immunophoresis in clearing free circulating RICs after adequate tumor uptake has severe drawbacks and limitations. For example, studies using doses of anti-Lym-1 antibodies directly iodinated with ¹³¹I demonstrated that although a significant portion of the radiolabeled antibody can be removed by immunophoresis of a patient's plasma through an anti-gammaglobulin column, the process is cumbersome, expensive, and the anti-gammaglobulin column has not been readily available (DeNardo et al., J. of Nucl. Med., 32:921 (1991); DeNardo et al., J. of Nucl. Med., 34:1020-1027 (1993)). Thus, there is no current means for effective and efficient removal of free circulating RICs from a patient's plasma.

In 1975, the same year that MAb technology was invented, Ringsdorf (J. Polymer Sci. Polymer Symp., 51:135 (1975)) proposed the use of biodegradable spacers/linkers for targeted drug release. Since the inception of antibody-targeted therapy, linking agents have been considered a key ingredient of drug development. An optimal linker should: (1) link the targeting agent to the biological agent without impairing the functionality of either; (2) retain a stable linkage in the circulation; and (3) degrade under specified conditions. More particularly, specific cleavage sites in the linker between the radionuclide-bound chelating agent (“radio-chelate”) and the MAb have been developed in an attempt to create rapid elimination of the radio-chelate from normal liver cells and kidney cells. Biodegradable linkers explored for reducing radiation to normal tissues or organs from RIT include esters, thioesthers, disulfides, amides, and hydrocarbon chains (Peterson and Meares, Bioconj. Chem., 10:553-557 (1999); Faivre-Chauvet et al., Nucl. Med. Biol., 20:763-771 (1993); Quadri et al., J. Nucl. Med., 34:938-945 (1993); Haseman et al., Eur. J. Nucl. Med., 12:455-460 (1986); Arano et al., Bioconj. Chem., 9:497-506 (1998)). Peptide linkers are preferred because of their potential stability in the circulation. In fact, simple tetrapeptides have been shown to be degraded by endoproteases in vitro at enzyme levels present in vivo (Peterson and Meares, Bioconj. Chem., 10:553-557 (1999); Studer et al., Bioconj. Chem., 3:424 (1992); Li and Meares, Bioconj. Chem., 4:275-283 (1993)). However, none of these linkers provides an RIC that is both selectively cleaved under specified conditions and stable in the circulation, i.e., resistant to proteases found in plasma and from tumor cells.

Thus, there is a need to develop a tumor-targeting agent such as an RIC that (1) binds to the tumor with high specificity and avidity, i.e., a high therapeutic index; (2) is resistant to cleavage or degradation from proteases found in plasma or tumor cells; (3) is selectively cleaved by the administration of an exogenous protease; and (4) is rapidly removed from normal tissues and organs following protease cleavage. The present invention satisfies this and other needs.

### BRIEF SUMMARY OF THE INVENTION

The present invention provides novel compositions and methods for improving site-specific delivery of biological agents to the site of a tumor and enhancing removal of those agents from normal tissues and organs.

In one aspect, the present invention provides a conjugate comprising:
(i) a biological agent selected from the group consisting of a therapeutic agent, an imaging agent, and mixtures thereof;
(ii) a targeting moiety; and
(iii) a linking group covalently attaching the biological agent to the targeting moiety, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein the linking group is selectively cleaved by a protease.

In another aspect, the present invention provides a method for treating cancer in a subject in need thereof, the method comprising:
(a) administering to the subject a conjugate comprising an effective anticancer agent covalently attached to a targeting moiety by a cleavable linking group, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, wherein the linking group is stable in plasma and is selectively cleaved by a protease; and
(b) administering to the subject an amount of the protease effective to increase the release of unbound anticancer agent relative to the amount of release of the unbound anticancer agent in the absence of the protease.

In yet another aspect, the present invention provides a method for imaging a tumor, organ, or tissue, the method comprising:
(a) administering to a subject in need of such imaging, a conjugate comprising an imaging agent covalently attached via a linking group to a targeting moiety specific for the tumor, organ, or tissue, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an a-amino acid having an L-configuration, and wherein the linking group is selectively cleaved by a protease;
(b) detecting radiation from the imaging agent to determine where the conjugate is concentrated in the subject; and
(c) administering to the subject a protease that selectively cleaves the linking group to increase clearance of unbound imaging agent from the subject.

In still yet another aspect, the present invention provides a kit for radiotherapy comprising:
(a) a first container holding a radioimmunoconjugate having a radiopharmaceutical attached via a linking group to a targeting antibody or antibody fragment, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein the linking group contains a cleavage site recognized by a co-administered protease;
(b) a second container holding the co-administered protease; and
(c) directions for use of the radioimmunoconjugate and the co-administered protease in radiotherapy.

In a further aspect, the present invention provides a kit for radioimaging comprising:
(a) a first container holding a radioimmunoconjugate having a radiopharmaceutical attached via a linking group to a targeting antibody or antibody fragment, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein the linking group contains a cleavage site recognized by a co-administered protease;
(b) a second container holding the co-administered protease; and
(c) directions for use of the radioimmunoconjugate and the co-administered protease in radioimaging.

Other objects, features, and advantages of the present invention will be apparent to one of skill in the art from the following detailed description and figures.

### BRIEF DESCRIPTION OF THE DRAWINGS

FIG. 1 shows the activity of plasma and TNKase® on peptides at different time points (hours). The left graph shows the Fluorescence intensity of the peptides in plasma alone and the right graph shows the Fluorescence intensity of the peptides in plasma containing 10 μg/ml TNKase®.

FIG. 2 shows the stability of peptides #17, #19, and #20, as well as the PFGRSA peptide, in supernatants collected from several tumor cell line cultures.

FIG. 3 shows the schemes for the synthesis of DOTA-peptide-antibody conjugates of the present invention.

FIG. 4 shows the mass spectrometric analysis of a ChL6-rqYKYkf-DOTA conjugate of the present invention.

FIG. 5 shows the radiograms of an ¹¹¹In-labeled ChL6-rqYKYkf-DOTA conjugate digested with 10 μg/ml or 1 mg/ml TNKase®. In FIG. 5A, the conjugate was incubated with human plasma alone (closed circle) or with TNKase® at 10 μg/ml for 2 hours (closed square) or 3 days (open circle). In FIG. 5B, the conjugate was incubated with human plasma alone (closed circle) or with TNKase® at 1 mg/ml for 2 hours (open circle).

### DETAILED DESCRIPTION OF THE INVENTION

I. Definitions

As used herein, the following terms have the meanings ascribed to them unless specified otherwise.

The term “conjugate” refers to a chemical compound that has been formed by the joining or attachment of two or more compounds. In particular, a conjugate of the present invention comprises a biological agent covalently attached via a linking group to a targeting moiety.

The term “immunoconjugate” refers to a composition comprising an antibody attached to a second molecule such as a detectable label or effector molecule. The antibody may be linked to the second molecule by covalent linkage through the use of a linking group. Representative effector molecules include biological agents, therapeutic agents, imaging agents, anticancer agents, and the like.

The terms “radioimmunoconjugate” and “RIC” are used interchangeably herein to refer to a composition comprising an antibody attached to a radiopharmaceutical. The antibody may be linked to the radiopharmaceutical by covalent linkage through the use of a linking group.

The terms “radioimmunotherapy,” “RIT,” and “radiotherapy” are used interchangeably herein to refer to the ability to treat certain types of tumors using radionuclides conjugated to antibodies directed against tumor antigens.

The term “biological agent” refers to a chemical substance, such as a small molecule, macromolecule, or metal ion, that either causes an observable change in the structure, function, or composition of a cell or permits direct visualization of the cell, upon binding to the cell surface and/or uptake by the cell. Observable changes include increased or decreased expression of one or more mRNAs, increased or decreased expression of one or more proteins, phosphorylation of a protein or other cell component, inhibition or activation of an enzyme, inhibition or activation of binding between members of a binding pair, an increased or decreased rate of synthesis of a metabolite, increased or decreased cell proliferation, and the like.

Included within this definition are “therapeutic agents,” which refer, without limitation, to any composition that can be used to the benefit of a mammalian species. Such agents may take the form of ions, small organic molecules, peptides, proteins, or polypeptides, and oligosaccharides. Also included within this definition are “imaging agents,” which refer, without limitation, to any composition that can be used to directly visualize and/or localize a cell or group of cells in a mammalian species. Such agents may take the form of ions, small organic molecules, peptides, proteins, or polypeptides, and oligosaccharides.

The term “targeting moiety” refers to species that will selectively localize in a particular tumor, tissue, organ, or other region of the body. The localization is mediated by specific recognition of molecular determinants, molecular size of the targeting agent or conjugate, ionic interactions, hydrophobic interactions, and the like. Other mechanisms of targeting an agent to a particular tissue or region are known to those of skill in the art. Exemplary targeting moieties include antibodies, antibody fragments, small organic molecules, peptides, peptoids, proteins, polypeptides, oligosaccharides, transferrin, HS-glycoprotein, coagulation factors, serum proteins, β-glycoprotein, G-CSF, GM-CSF, M-CSF, EPO, and the like. The somatostatin analogue D-phe(1)-tyr(3)-octreotide is an example of a peptide suitable for use as a targeting moiety.

The term “radiopharmaceutical” refers to a radioactive compound used for therapeutic, imaging, or diagnostic purposes in a mammalian subject. Suitable radiopharmaceuticals include, but are not limited to, radionuclides, radionuclides directly coupled to an antibody, radionuclides directly coupled to a linking group, and radionuclides bound to a chelating agent (“radio-metal chelate”). The compound Bexxar® is an example in which the radionuclide is directly coupled to an antibody. The compound Zevalin® is an example in which the radionuclide is present as a radio-metal chelate conjugated to an antibody. As used herein, a radionuclide directly coupled to a linking group refers to a linking group that has been radiolabeled (e.g., radioiodinated) with a radionuclide by means of chemical coupling or other methods known to one of skill in the art. Preferably, the radiopharmaceutical of the present invention comprises a radionuclide bound to a chelating agent.

As used herein, a “chelating agent” refers to a compound which binds to a metal ion, such as a radionuclide, with considerable affinity and stability. In addition, the chelating agents of the present invention are bifunctional, having a metal ion chelating group at one end and a reactive functional group capable of binding to peptides, polypeptides, or proteins at the other end. Suitable bifunctional chelating agents include, but are not limited to, 1,4,7,10-tetraazacyclododecane-N,N′,N″,N′″-tetraacetic acid (DOTA), a bromoacetamidobenzyl derivative of DOTA (BAD), 1,4,8,11-tetraazacyclotetradecane-N,N′,N″,N′41 -tetraacetic acid (TETA), diethylenetriaminepentaacetic acid (DTPA), the dicyclic dianhydride of diethylenetriaminepentaacetic acid (ca-DTPA), 2-(p-isothiocyanatobenzyl)diethylenetriaminepentaacetic acid (SCNBzDTPA), and 2-(p-isothiocyanatobenzyl)-5(6)-methyl-diethylenetriaminepentaacetic acid (MxDTPA) (see, Ruegg et al., Cancer Research, Vol. 50: 14 4221-4226, 1990; DeNardo et al., Clinical Cancer Research, Vol. 4: 10 2483-2490, 1998). Other chelating agents include EDTA, NTA, HDTA and their phosphonate analogs such as EDTP, HDTP, NTP (see, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989; and references contained therein).

The term “radionuclide” refers to a nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance. Radionuclides suitable for use in the present invention include, but are not limited to, fluorine 18 (¹⁸F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y), rhodium 105 (¹⁰⁵Rh), silver 111 (¹¹¹Ag), indium 111 (¹¹¹In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹Tl), astatine 211 (²¹¹At), and bismuth 212 (²¹²Bi). As used herein, the “m” in ^{117m}Sn and ^{99m}Tc stands for meta state. Additionally, naturally occurring radioactive elements such as uranium, radium, and thorium, which typically represent mixtures of radioisotopes, are suitable examples of radionuclides. ⁶⁷Cu, ¹³¹I, ¹⁷⁷Lu, and ¹⁸⁶Re are beta- and gamma-emitting radionuclides. ²¹²Bi is an alpha- and beta-emitting radionuclide. ²¹¹At is an alpha-emitting radionuclide. ³²P, ⁴⁷Sc, ⁸⁹Sr, ⁹⁰Y, ¹⁰⁵Rh, ¹¹¹Ag, ^{117m}Sn, ¹⁴⁹Pm, ¹⁵³Sm, ¹⁶⁶Ho, and ¹⁸⁸Re are examples of beta-emitting radionuclides. ⁶⁷Ga, ¹¹¹In, ^{99m}Tc, and ²⁰¹Tl are examples of gamma-emitting radionuclides. ⁵⁵Co, ⁶⁰Cu, ⁶¹Cu, ⁶²Cu, ⁶⁶Ga, ⁶⁸Ga, ⁸²Rb, and ⁸⁶Y are examples of positron-emitting radionuclides. ⁶⁴Cu is a beta- and positron-emitting radionuclide.

An “anticancer agent” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radiopharmaceuticals. Also encompassed within the scope of the term “anticancer agent” are conjugates of peptides with anti-tumor activity, e.g. TNF-α. Conjugates include, but are not limited to, those formed between an anticancer agent and a targeting moiety by covalent attachment via a linking group. A representative conjugate is that formed between a radiopharmaceutical and an antibody by covalent attachment via a peptide linking group.

The term “amino acid” refers to naturally occurring α-amino acids and their stereoisomers, as well as unnatural amino acids such as amino acid analogs, amino acid mimetics, synthetic amino acids, β-amino acids, γ-amino acids, and N-substituted glycines in either the L- or D-configuration that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Stereoisomers” of naturally occurring amino acids refers to mirror image isomers of the naturally occurring amino acids, such as D-amino acids. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. In β-amino acids, the amino group is bonded to the β-carbon atom of the carboxyl group such that there are two carbon atoms between the amino and carboxyl groups. In γ-amino acids, the amino group is bonded to the γ-carbon atom of the carboxyl group such that there are three carbon atoms between the amino and carboxyl groups. Suitable side chains (e.g., R groups) for β- or γ-amino acids include, but are not limited to, side chains present in naturally occurring amino acids and unnatural amino acids such as amino acid analogs, amino acid mimetics, synthetic amino acids, and N-substituted glycines.

The term “N-substituted glycine” refers to a glycine amino acid where an amino acid side chain is attached to the glycine nitrogen atom. Suitable amino acid side chains (e.g., R groups) include, but are not limited to, side chains present in naturally occurring amino acids and side chains present in unnatural amino acids such as amino acid analogs, amino acid mimetics, synthetic amino acids, β-amino acids, and γ-amino acids. Examples of N-substituted glycines suitable for use in the present invention include, without limitation, N-(2-aminoethyl)glycine, N-(3-aminopropyl)glycine, N-(2-methoxyethyl)glycine, N-benzylglycine, (S)-N-(1-phenylethyl)glycine, N-cyclohexylmethylglycine, N-(2-phenylethyl)glycine, N-(3-phenylpropyl)glycine, N-(6-aminogalactosyl)glycine, N-(2-(3′-indolylethyl)glycine, N-(2-(p-methoxyphenylethyl))glycine, N-(2-(p-chlorophenylethyl)glycine, and N-[2-(p-hydroxyphenylethyl)]glycine. Such N-substituted glycines can have an L- or D-configuration. N-substituted glycine oligomers, referred to herein as “peptoids,” have been shown to be protease resistant (Miller et al., Drug Dev. Res., 35:20-32 (1995)). As such, a peptoid linker containing at least one α-amino acid having an L-configuration is within the scope of the present invention.

Amino acids may be referred to herein by either their commonly known three letter symbols or by the one-letter symbols recommended by the IUPAC-IUB Biochemical Nomenclature Commission.

With respect to amino acid sequences, one of skill will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acids include, but are not limited to, naturally occurring amino acids such as α-amino acids having an L-configuration, sterorisomers of naturally occurring amino acids such as α-amino acids having a D-configuration, and unnatural amino acids such as amino acid analogs, amino acid mimetics, synthetic amino acids, β-amino acids, and γ-amino acids, in either the L- or D-configuration. For example, the unnatural amino acids of Liu and Lam (Anal. Biochem., 295:9-16 (2001)) are suitable for use in the present invention.

Conservative substitution tables providing functionally similar amino acids are well known in the art. For example, substitutions may be made wherein an aliphatic amino acid (G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, E or D, may be substituted with its uncharged counterpart, Q or N, respectively; or vice versa. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins, 1984).

The term “peptide” refers to a compound made up of a single chain of D- or L-amino acids or a mixture of D- and L-amino acids joined by peptide bonds. Generally, peptides contain at least two amino acid residues and are less than about 50 amino acids in length. Preferably, the peptides of the present invention are between four and eight amino acids in length. D-amino acids are represented herein by a lower-case one-letter amino acid symbol (e.g., r for D-arginine), whereas L-amino acids are represented by an upper case one-letter amino acid symbol (e.g., R for L-arginine).

The term “protein” refers to a compound that is composed of linearly arranged amino acids linked by peptide bonds, but in contrast to peptides, has a well-defined conformation. Proteins, as opposed to peptides, generally consist of chains of 50 or more amino acids.

The term “polypeptide” refers to a polymer of at least two amino acid residues and which contains one or more peptide bonds. “Polypeptide” encompasses peptides and proteins, regardless of whether the polypeptide has a well-defined conformation.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The remainder of each chain defines a constant region that is conserved and exhibits low variability among different antibodies. Each light chain contains one constant region (C_{L}) and each heavy chain contains three constant regions (C_{H}1, C_{H}2, C_{H}3). Different classes of constant regions in the stem of the antibody generate different isotypes with differing properties based on their amino acid sequence.

Antibodies exist, e.g., as intact immunoglobulins or as a number of well-characterized fragments produced by digestion with various peptidases, referred to herein as “antibody fragments.” Thus, for example, pepsin digests an antibody below the disulfide linkages in the hinge region to produce F(ab)′₂, a dimer of Fab (fragment, antigen binding) which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see, Fundamental Immunology, Paul ed., 3d ed., 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g., recombinant, monoclonal, or polyclonal antibodies, any technique known in the art can be used (see, e.g., Kohler & Milstein, Nature 256:495-497 (1975); Kozbor et al., Immunology Today, 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,816,567 and 4,946,778) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016; Marks et al., Biotechnology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); and Lonberg & Huszar, Intern. Rev. Immunol., 13:65-93 (1995)). Alternatively, phage display technology can be used to identify antibodies and heteromeric Fab fragments that specifically bind to selected antigens (see, e.g., McCafferty et al., Nature, 348:552-554 (1990); Marks et al., Biotechnology, 10:779-783 (1992)). Antibodies can also be made bispecific, i.e., able to recognize two different antigens (see, e.g., WO 93/08829; Traunecker et al., EMBO J., 10:3655-3659 (1991); and Suresh et al., Methods in Enzymology, 121:210 (1986)). Antibodies can also be heteroconjugates, e.g., two covalently joined antibodies, or immunotoxins (see, e.g., U.S. Pat. No. 4,676,980; WO 91/00360; WO 92/200373; and EP 03089).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a chimeric antibody can comprise mouse protein sequence in the variable region and human protein sequence in the constant region. A “humanized antibody” comprises even fewer mouse protein sequence in the variable region than chimeric antibodies. Such mouse protein sequence has been replaced by human protein sequence.

Monoclonal antibodies (MAbs) suitable for use in the present invention include, but are not limited to, ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibodies. As used herein, “ChL6 antibody” refers to an anti-adenocarcinoma chimeric L6 MAb that reacts with an integral membrane glycoprotein found on some tumor cells. “Lym-1 antibody” refers to an anti-lymphoma MAb that recognizes a cell surface antigen on malignant B cells. “CD1b antibody” refers to an anti-CD1b MAb such as the IgG1 T101 MAb used for detection or treatment of cutaneous T-cell lymphomas. T3 is an example of an anti-CD3 IgG1 MAb antibody. T1 is an example of an anti-CD5 IgG2a MAb. My4 is an example of an anti-CD14 IgG2b MAb. NKH1 is an example of an anti-CD56 IgG1 MAb. “CD20 antibody” refers to an anti-CD20 MAb that binds to the CD20 antigen found on normal and malignant B lymphomas. Rituximab is an example of an anti-CD20 chimeric IgG1 MAb available from IDEC Pharmaceuticals. Tositumomab, ibritumomab, B1, and 2B8 are other examples of anti-CD20 MAbs. “CD22 antibody” refers to an anti-CD22 MAb such as epratuzumab or LL2 that binds to the CD22 antigen. “CD33 antibody” refers to an anti-CD33 MAb that binds to the CD33 antigen found on monocytes, activated T cells, granulocytes, myeloid progenitors, and mast cells. Gemtuzumab ozogamicin is a humanized anti-CD33 IgG4 MAb conjugated to calicheamicin, a complex oligosaccharide that makes double-stranded breaks in DNA, and is available from Wyeth Laboratories. M195, an example of an anti-CD33 MAb, is a murine IgG2a MAb or humanized IgG1 MAb that binds to the CD33 antigen. “CD52 antibody” refers to an anti-CD52 MAb that binds to the CD52 antigen found on normal and malignant B and T lymphocytes and additional white blood cells. Alemtuzumab is an example of an anti-CD52 humanized IgG1 MAb available from ILEX Pharmaceuticals. “HER2/neu antibody” refers to an anti-HER2 MAb that binds to HER2, a growth factor receptor found on some tumor cells. Trastuzumab is an example of an anti-HER2 MAb available from Genentech. The anti-Tac MAb specific for the IL-2R α-chain is an example of an IL-2R antibody. “EGF receptor antibody” refers to an anti-EGF receptor MAb that binds to the EGF receptor found on tumor cells. Cetuximab is an example of an anti-EGF receptor MAb available from ImClone Systems and Bristol-Myers Squibb. Anti-tenascin antibodies include, without limitation, BC-2 and 81C6 MAbs. Anti-TAG-72 antibodies include, without limitation, CC49 and B72.3 MAbs. Other antibodies suitable for use in the present invention include, but are not limited to, m170, BrE-3, hMN-14 (humanized anti-CEA Ab), 3F8, HMFG-1, HMFG-2, AUA1, H317, and H17E2.

The term “protease” refers to any of various enzymes that catalyze the degradation of peptides, polypeptides, and proteins by hydrolyzing at least one of their peptide bonds. Suitable proteases for use in the present invention include, but are not limited to, endopeptidases (e.g., serine proteases and metalloproteases) and exopeptidases (e.g., carboxypeptidases and aminopeptidases). In particular, proteases such as cathepsin B, cathepsin D, trypsin, chymotrypsin, and pepsin are suitable for use in the present invention. Preferably, the protease is tissue-type plasminogen activator (t-PA) or a modified form thereof. t-PA is a clot-dissolving serine protease produced naturally by cells in the walls of blood vessels and catalyzes the conversion of plasminogen to plasmin. Modified forms of t-PA include Activase® and TNKase®, both currently produced by and commercially available from Genentech. The amino acid sequence of TNKase® is identical to Activase® except for a substitution of threonine 103 with asparagine, a substitution of asparagine 117 with glutamine, and a substitution within the protease domain of amino acids 296-299 with four alanines. Activase® has an initial plasma half-life of 5 minutes whereas TNKase® has an initial plasma half-life of 20-25 minutes.

The term “selectively cleaved” refers to the hydrolysis of a peptide bond by a protease upon recognition of a specific amino acid residue or amino acid sequence in a peptide, polypeptide, or protein. For example, trypsin selectively cleaves peptide bonds on the carboxyl-terminal side of lysine (K) and arginine (R) amino acid residues. Chymotrypsin selectively cleaves peptide bonds on the carboxyl-terminal side of phenylalanine (F), tryptophan (W), and tyrosine (Y) residues. t-PA selectively cleaves a single peptide bond between arginine (R) 560 and valine (V) 561 in plasminogen in vivo. This peptide bond is the only known physiological substrate for t-PA. However, in vitro studies have shown that t-PA is also capable of selectively cleaving peptides with the consensus sequence: X-(G>A)-R-X′-(A>G), wherein X is any amino acid, X′ is most often R, but could be other residues, and the sequence is selectively cleaved between R and X′ (Ding et al., Proc. Natl. Acad. Sci. USA, 92: 7627-7631, 1995; Coombs et al., J. of Biol. Chemistry, 273: 4323-4328, 1998). Preferably, the t-PA or modified form thereof used in the present invention selectively cleaves a peptide, polypeptide, or protein at the carboxyl-terminal side of lysine (K) or arginine (R) residues, including stereoisomers, analogs, mimetics, and conservatively modified variants thereof.

The term “cancer” refers to any of various malignant neoplasms characterized by the proliferation of anaplastic cells that tend to invade surrounding tissue and metastasize to new body sites. Examples of different types of cancer suitable for treatment using the present invention include, but are not limited to, lung cancer, breast cancer, bladder cancer, thyroid cancer, liver cancer, pleural cancer, pancreatic cancer, ovarian cancer, cervical cancer, testicular cancer, colon cancer, B-cell lymphoma, non-Hodgkin's lymphoma, Burkitt's lymphoma, fibrosarcoma, neuroblastoma, glioma, melanoma, monocytic leukemia, and myelogenous leukemia.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to a subject. Adminsitration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

II. General Overview

The present invention provides novel compositions and methods for improving the targeted delivery of biological agents to the site of a tumor while reducing the exposure of normal tissues and organs to those agents by enhancing their removal and clearance from the plasma of a subject.

The present invention is based on the discovery that an FDA-approved drug can be used to selectively cleave a linking group between a radionuclide-chelating agent (“radio-metal chelate”) and an antibody molecule so that free radio-metal chelate can be rapidly cleared through the kidney after adequate radionuclide uptake by the tumor. The linking group is referred to as an “on-demand cleavable” linker (ODC-linker) because its selective cleavage is controlled by administration of the FDA-approved drug, although the present invention is not limited to the use of FDA-approved drugs. Importantly, the ODC-linker is resistant to cleavage by proteases found in human plasma or tumor cell culture supernatants, and is therefore specifically cleaved upon administration of the FDA-approved drug. As a result, the blood clearance of the radio-metal chelate is generally better than that observed using immunopheresis, the uptake of radionuclide by the tumor is not compromised, and the cumbersome and expensive method of immunophoresis is avoided.

III. Description of the Embodiments

In one aspect, the present invention provides a conjugate comprising:
(i) a biological agent selected from the group consisting of a therapeutic agent, an imaging agent, and mixtures thereof;
(ii) a targeting moiety; and
(iii) a linking group covalently attaching the biological agent to the targeting moiety, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein the linking group is selectively cleaved by a protease.

In one embodiment, the biological agent is a therapeutic agent. In another embodiment, the therapeutic agent is a radiopharmaceutical. In a preferred embodiment, the radiopharmaceutical is a radionuclide bound to a chelating agent. In another preferred embodiment, the linking group is radiolabeled with a radionuclide. Suitable radionuclides include, but are not limited to, ⁴⁷Sc, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y ⁸⁹Sr, ⁹⁰Y, ¹⁰⁵Rh, ¹¹¹Ag, ¹¹¹In, ^{117m}Sn, ^{99m}Tc, ¹⁴⁹Pm, ¹⁵³Sm, ¹⁶⁶Ho, ¹⁷⁷Lu, ¹⁸⁶Re, ¹⁸⁸Re, ²⁰¹Tl, ²¹¹At, ²¹²Bi, and mixtures thereof. Preferably, the linking group is radiolabeled with ¹⁸F, ¹²⁴I, ¹²⁵I, ¹³¹I, or mixtures thereof. Preferably, the radionuclide bound to a chelating agent is ⁹⁰Y. Suitable chelating agents include, but are not limited to, DOTA, BAD, TETA, DTPA, EDTA, NTA, HDTA, their phosphonate analogs, and mixtures thereof. Preferably, the chelating agent binding the radionuclide is DOTA. In a particularly preferred embodiment, the radiopharmaceutical is ⁹⁰Y bound to DOTA. In another particularly preferred embodiment, the radiopharmaceutical is ⁶⁴Cu or ¹¹¹In bound to DOTA.

In another embodiment, the targeting moiety is selected from the group consisting of an antibody, antibody fragment, small organic molecule, peptide, protein, polypeptide, glycoprotein, oligosaccharide, and the like. In a preferred embodiment, the targeting moiety is an antibody or antibody fragment. Suitable antibodies include, but are not limited to, monoclonal, polyclonal, and recombinant antibodies, as well as antigen binding fragments (Fab) thereof. Preferably, the antibody is a monoclonal antibody. The antibodies of the present invention can be derived from any mammalian species, such as mouse, rat, and rabbit, and are preferably humanized or chimeric, for example, by including human protein sequence in the constant region of the antibody light and heavy chains. In another embodiment, the antibody binds to an antigen on the surface of a cancer or tumor cell. Such antibodies include, but are not limited to, antibodies against the ChL6, Lym-1, CD1b, CD3, CD5, CD 14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antigens.

In yet another embodiment, the linking group covalently attaching the biological agent to the targeting moiety is selected from the group consisting of peptides, peptoids, polypeptides, and proteins. In a preferred embodiment, the linking group is a peptide. Suitable amino acids for use in the linking group include, but are not limited to, naturally occurring α-amino acids (e.g., α-amino acids having an L-configuration), their stereoisomers (e.g., α-amino acids having a D-configuration), and unnatural amino acids such as amino acid analogs, amino acid mimetics, synthetic amino acids, β-amino acids, γ-amino acids, and N-substituted glycines. Preferably, the amino acids are α-amino acids having a D-configuration or an L-configuration. In a further embodiment, the linking group is a modified peptoid containing at least one α-amino acid having an L-configuration.

In still yet another embodiment, the linking group comprises from three to twenty amino acids. In a preferred embodiment, the linking group comprises from two to eight amino acids having a D-configuration and from two to eight amino acids having an L-configuration, wherein at least one amino acid having an L-configuration is an α-amino acid. In another preferred embodiment, the linking group comprises two amino acids having a D-configuration and three amino acids having an L-configuration, wherein at least one amino acid having an L-configuration is an α-amino acid. In a particularly preferred embodiment, the linking group comprises four α-amino acids having a D-configuration and three α-amino acids having an L-configuration. In another particularly preferred embodiment, the linking group comprises a heptapeptide having the sequence -rqYKYkf- (SEQ ID NO:1), wherein the lower-case one-letter amino acid symbol represents an α-amino acid having a D-configuration and the upper-case one-letter amino acid symbol represents an α-amino acid having an L-configuration. Conservatively modified variants of such amino acid sequences are also within the scope of the present invention.

In a further embodiment, the protease that selectively cleaves the linking group is selected from the group consisting of endopeptidases, serine proteases, metalloproteases, exopeptidases, carboxypeptidases, aminopeptidases, and the like. More particularly, proteases such as tissue-type plasminogen activator (t-PA), cathepsin B, cathepsin D, trypsin, chymotrypsin, and pepsin are suitable for use in the present invention. Preferably, the protease is t-PA or a modified form thereof. In preferred embodiments, the protease is a modified form of t-PA, such as Activase® or TNKase®. In a particularly preferred embodiment, the protease is TNKase®.

In a preferred embodiment, the present invention provides a conjugate, wherein the biological agent is a radiopharmaceutical, the targeting moiety is a monoclonal antibody, and the linking group comprises a heptapeptide having the sequence -rqYKYkf- (SEQ ID NO:1) or a conservatively modified variant thereof, wherein the linking group is selectively cleaved by TNKase®. In one embodiment, the radiopharmaceutical is a radionuclide bound to a chelating agent. In a second embodiment, the linking group is radiolabeled with a radionuclide. In a third embodiment, the radionuclide is selected from the group consisting of ⁴⁷Sc, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁸⁹Sr, ⁹⁰Y, ¹⁰⁵Rh, ¹¹¹Ag, ¹¹¹In, ^{117m}Sn, ^{99m}Tc, ¹⁴⁹Pm, ¹⁵³Sm, ¹⁶⁶Ho, ¹⁷⁷Lu, ¹⁸⁶Re, ¹⁸⁸Re, ²⁰¹Tl, ²¹¹At, ²¹²Bi, and mixtures thereof. In a fourth embodiment, the linking group is radiolabeled with ¹⁸F, ¹²⁴I, ¹²⁵I, ¹³¹I, or mixtures thereof. In a fifth embodiment, the chelating agent is DOTA. Preferably, the radiopharmaceutical is ⁹⁰Y bound to DOTA. In a sixth embodiment, the monoclonal antibody is selected from the group consisting of anti-ChL6, Lym-l, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibodies. In a particularly preferred embodiment, the biological agent is ¹¹¹In bound to DOTA, the targeting moiety is an anti-ChL6 antibody, and the linking group comprises a heptapeptide having the sequence -rqYKYkf- (SEQ ID NO:1), wherein the linking group is selectively cleaved by TNKase®.

In another aspect, the present invention provides a method for treating cancer in a subject in need thereof, the method comprising:
(a) administering to the subject a conjugate comprising an effective anticancer agent covalently attached to a targeting moiety by a cleavable linking group, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, wherein the linking group is stable in plasma and is selectively cleaved by a protease; and
(b) administering to the subject an amount of the protease effective to increase the release of unbound anticancer agent relative to the amount of release of the unbound anticancer agent in the absence of the protease.

In one embodiment, the anticancer agent is selected from the group consisting of cytotoxins and agents such as antimetabolites, alkylating agents, anthracyclines, antibiotics, antimitotic agents, procarbazine, hydroxyurea, asparaginase, corticosteroids, interferons, radiopharmaceuticals, and peptides. In a preferred embodiment, the anticancer agent is a radiopharmaceutical. In another embodiment, the radiopharmaceutical is a radionuclide bound to a chelating agent. In yet another embodiment, the linking group is radiolabeled with a radionuclide. In still yet another embodiment, the radionuclide is selected from the group consisting of⁴⁷Sc, ⁶⁴Cu, ⁶⁷Cu, ⁸⁶Y, ⁸⁷Y, ⁸⁹Sr, ⁹⁰Y, ¹⁰⁵Rh, ¹¹¹Ag, ¹¹¹In, ^{117m}Sn, ¹⁴⁹Pm, ¹⁵³Sm, 166Ho, 177Lu, ¹⁸⁶Re, ¹⁸⁸Re, ²¹¹At, ²¹²Bi, and mixtures thereof. Preferably, the linking group is radiolabeled with ¹⁸F, ¹²⁴I, ¹²⁵I, ¹³¹I, or mixtures thereof. In a further embodiment, the chelating agent is DOTA. Preferably, the radiopharmaceutical is ⁹⁰Y bound to DOTA.

In another embodiment, the targeting moiety is selected from the group consisting of an antibody, antibody fragment, small organic molecule, peptide, protein, polypeptide, glycoprotein, oligosaccharide, and the like. In a preferred embodiment, the targeting moiety is an antibody or antibody fragment. In a particularly preferred embodiment, the antibody or antibody fragment is a monoclonal antibody. In a further embodiment, the monoclonal antibody is selected from the group consisting of anti-ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibodies.

In yet another embodiment, the linking group covalently attaching the biological agent to the targeting moiety is selected from the group consisting of peptides, peptoids, polypeptides, and proteins. In a preferred embodiment, the linking group is a peptide. Most preferably, the linking group is stable in the plasma of a subject, and is resistant to cleavage or degradation by urokinase, matrilysin, or other proteases found in plasma or at the tumor site. Suitable amino acids for use in the linking group include, but are not limited to, naturally occurring α-amino acids (e.g., α-amino acids having an L-configuration), their stereoisomers (e.g., α-amino acids having a D-configuration), and unnatural amino acids such as amino acid analogs, amino acid mimetics, synthetic amino acids, β-amino acids, γ-amino acids, and N-substituted glycines. Preferably, the amino acids are α-amino acids having a D-configuration or an L-configuration. In a further embodiment, the linking group is a modified peptoid containing at least one α-amino acid having an L-configuration.

In still yet another embodiment, the linking group comprises from three to twenty amino acids. In a preferred embodiment, the linking group comprises from two to eight amino acids having a D-configuration and from two to eight amino acids having an L-configuration, wherein at least one amino acid having an L-configuration is an α-amino acid. In another preferred embodiment, the linking group comprises two amino acids having a D-configuration and three amino acids having an L-configuration, wherein at least one amino acid having an L-configuration is an α-amino acid. In a particularly preferred embodiment, the linking group comprises four α-amino acids having a D-configuration and three α-amino acids having an L-configuration. In another particularly preferred embodiment, the linking group comprises a heptapeptide having the sequence -rqYKYkf- (SEQ ID NO:1), wherein the lower-case one-letter amino acid symbol represents an α-amino acid having a D-configuration and the upper-case one-letter amino acid symbol represents an α-amino acid having an L-configuration. Conservatively modified variants of such amino acid sequences are also within the scope of the present invention.

In a further embodiment, the protease that selectively cleaves the linking group is selected from the group consisting of endopeptidases, serine proteases, metalloproteases, exopeptidases, carboxypeptidases, aminopeptidases, and the like. More particularly, proteases such as tissue-type plasminogen activator (t-PA), cathepsin B, cathepsin D, trypsin, chymotrypsin, and pepsin are suitable for use in the present invention. Preferably, the protease is t-PA or a modified form thereof. In preferred embodiments, the protease is a modified form of t-PA, such as Activase® or TNKase®. In a particularly preferred embodiment, the protease is TNKase®.

Administration of the protease may occur either at the same time as RIC administration, or the protease and RIC may be administered sequentially in a predetermined order. In a preferred embodiment, the protease is administered to a subject after the RIC is administered. The time of protease administration following RIC administration, or “intervention time,” is influenced by a number of factors, such as blood clearance rates, tumor uptake and clearance rates, and radionuclide decay rates. Preferably, the intervention time is between 2 and 24 hours. More preferably, the intervention time is at about 6 hours. The intervention time should be such that the protease increases the release of the chelated radionuclide anticancer agent from the RIC relative to its release in the absence of the protease. In a preferred embodiment, TNKase® administration results in at least a 75% reduction of radionuclide concentration in the plasma of a subject relative to control subjects.

In another preferred embodiment, the present invention provides a conjugate, wherein the anticancer agent is a radiopharmaceutical, the targeting moiety is a monoclonal antibody, and the linking group comprises a heptapeptide having the sequence -rqYKYkf- (SEQ ID NO:1) or a conservatively modified variant thereof, wherein the linking group is selectively cleaved by TNKase®. Suitable radiopharmaceuticals and monoclonal antibodies are described above. In a particularly preferred embodiment, the anticancer agent is ¹¹¹In bound to DOTA, the targeting moiety is an anti-ChL6 antibody, and the linking group comprises a heptapeptide having the sequence -rqYKYkf- (SEQ ID NO:1), wherein the linking group is selectively cleaved by TNKase®.

In yet another aspect, the present invention provides a method for imaging a tumor, organ, or tissue, the method comprising:
(a) administering to a subject in need of such imaging, a conjugate comprising an imaging agent covalently attached via a linking group to a targeting moiety specific for the tumor, organ, or tissue, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an (-amino acid having an L-configuration, and wherein the linking group is selectively cleaved by a protease;
(b) detecting radiation from the imaging agent to determine where the conjugate is concentrated in the subject; and
(c) administering to the subject a protease that selectively cleaves the linking group to increase clearance of unbound imaging agent from the subject.

In one embodiment, the imaging agent is a radiopharmaceutical. In a preferred embodiment, the radiopharmaceutical is a radionuclide bound to a chelating agent. In another preferred embodiment, the linking group is radiolabeled with a radionuclide. Suitable radionuclides include, but are not limited to, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ^{99m}Tc, ²⁰¹Tl, and mixtures thereof. Preferably, the radionuclide bound to a chelating agent is ⁶⁴Cu, ⁹⁰Y, ¹¹¹In, or mixtures thereof. In a further preferred embodiment, the linking group is radiolabeled with ¹²⁵I, ¹³¹I, or mixtures thereof. Suitable chelating agents include, but are not limited to, DOTA, BAD, TETA, DTPA, EDTA, NTA, HDTA, their phosphonate analogs, and mixtures thereof. Preferably, the chelating agent binding the radionuclide is DOTA. In a particularly preferred embodiment, the radiopharmaceutical is ⁹⁰Y bound to DOTA. In another particularly preferred embodiment, the radiopharmaceutical is ¹¹¹In bound to DOTA. In yet another particularly preferred embodiment, the radiopharmaceutical is ⁶⁴Cu bound to DOTA.

In another embodiment, the targeting moiety specific for the tumor, organ, or tissue is selected from the group consisting of an antibody, antibody fragment, small organic molecule, peptide, protein, polypeptide, glycoprotein, oligosaccharide, and the like. In a preferred embodiment, the targeting moiety is an antibody or antibody fragment. In a particularly preferred embodiment, the antibody or antibody fragment is a monoclonal antibody. In a further embodiment, the monoclonal antibody is selected from the group consisting of anti-ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibodies. In another preferred embodiment, the targeting moiety is specific for a tumor.

In yet another embodiment, the linking group covalently attaching the biological agent to the targeting moiety is selected from the group consisting of peptides, peptoids, polypeptides, and proteins. In a preferred embodiment, the linking group is a peptide. Most preferably, the linking group is stable in the plasma of a subject, and is resistant to cleavage or degradation by urokinase, matrilysin, or other proteases found in plasma or at the tumor site. Suitable amino acids for use in the linking group include, but are not limited to, naturally occurring α-amino acids (e.g., α-amino acids having an L-configuration), their stereoisomers (e.g., α-amino acids having a D-configuration), and unnatural amino acids such as amino acid analogs, amino acid mimetics, synthetic amino acids, β-amino acids, γ-amino acids, and N-substituted glycines. Preferably, the amino acids are α-amino acids having a D-configuration or an L-configuration. In a further embodiment, the linking group is a modified peptoid containing at least one α-amino acid having an L-configuration.

In still yet another embodiment, the linking group comprises from three to twenty amino acids. In a preferred embodiment, the linking group comprises from two to eight amino acids having a D-configuration and from two to eight amino acids having an L-configuration, wherein at least one amino acid having an L-configuration is an α-amino acid. In another preferred embodiment, the linking group comprises two amino acids having a D-configuration and three amino acids having an L-configuration, wherein at least one amino acid having an L-configuration is an α-amino acid. In a particularly preferred embodiment, the linking group comprises four α-amino acids having a D-configuration and three α-amino acids having an L-configuration. In another particularly preferred embodiment, the linking group comprises a heptapeptide having the sequence -rqYKYkf- (SEQ ID NO:1), wherein the lower-case one-letter amino acid symbol represents an α-amino acid having a D-configuration and the upper-case one-letter amino acid symbol represents an α-amino acid having an L-configuration. Conservatively modified variants of such amino acid sequences are also within the scope of the present invention.

In a further embodiment, the protease that selectively cleaves the linking group is selected from the group consisting of endopeptidases, serine proteases, metalloproteases, exopeptidases, carboxypeptidases, aminopeptidases, and the like. More particularly, proteases such as tissue-type plasminogen activator (t-PA), cathepsin B, cathepsin D, trypsin, chymotrypsin, and pepsin are suitable for use in the present invention. Preferably, the protease is t-PA or a modified form thereof. In preferred embodiments, the protease is a modified form of t-PA, such as Activase® or TNKase®. In a particularly preferred embodiment, the protease is TNKase®.

In a preferred embodiment, the present invention provides a method for imaging a tumor, organ, or tissue, wherein the imaging agent is a radiopharmaceutical, the targeting moiety is specific for a tumor, and the linking group comprises a heptapeptide having the sequence -rqYKYkf- (SEQ ID NO:1) or a conservatively modified variant thereof, and is selectively cleaved by TNKase. Suitable radiopharmaceuticals and targeting moieties specific for a tumor such as monoclonal antibodies are described above. In a particularly preferred embodiment, the imaging agent is ¹¹¹In bound to DOTA, the targeting moiety is an anti-ChL6 antibody, and the linking group comprises a heptapeptide having the sequence -rqYKYkf- (SEQ ID NO:1), wherein the linking group is selectively cleaved by TNKase®.

The method for imaging a tumor, organ, or tissue according to the present invention may further comprise co-administering a therapeutic agent, wherein the therapeutic agent is an anticancer agent covalently attached via a linking group to a targeting moiety. Preferably, the anticancer agent is a radiopharmaceutical comprising a radionuclide bound to a chelating agent. Most preferably, the anticancer agent is ⁹⁰Y bound to DOTA. Alternatively, the anticancer agent is a radiopharmaceutical comprising a linking group radiolabeled with a radionuclide. Preferably, the linking group is radiolabeled with ¹²⁵I, ¹³¹I, or mixtures thereof. The anticancer agent may be attached to the same, similar, or different targeting moiety as the imaging agent, via the same linking group or a conservatively modified variant thereof. Preferably, the anticancer agent is attached via a peptide linking group to an antibody or antibody fragment. However, the antibody or antibody fragment in the RIC containing the anticancer agent need not be identical to the antibody or antibody fragment in the RIC containing the imaging agent. Further, the linking group attaching the anticancer agent to the targeting moiety need not be identical to the linking group attaching the imaging agent to the targeting moiety.

In a preferred embodiment, the imaging agent is ⁶⁴Cu or ¹¹¹In bound to DOTA, the anticancer agent is ⁹⁰Y bound to DOTA or ¹³¹I directly coupled to a peptide linking group, and both agents are attached via the identical peptide linking group to the identical antibody or antibody fragment. In another preferred embodiment, the imaging agent is ⁶⁴Cu or ¹¹¹In bound to DOTA, the anticancer agent is ⁹⁰Y bound to DOTA or ¹³¹I directly coupled to a peptide linking group, and both agents are attached via the identical peptide linking group to different antibodies or antibody fragments. In yet another preferred embodiment, the imaging agent is ⁶⁴Cu or ¹¹¹In bound to DOTA, the anticancer agent is ⁹⁰Y bound to DOTA or ¹³¹I directly coupled to a peptide linking group, and both agents are attached via different peptide linking groups to different antibodies or antibody fragments. These RICs may be co-administered to a subject in need thereof for both therapeutic and imaging purposes. Alternatively, these RICs may be administered sequentially in a predetermined order. Depending on the peptide linking group(s) used, one or more proteases may then be administered to the subject to increase the clearance of the imaging agent and/or the anticancer agent from the subject.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from an imaging agent of the present invention. Positron emission tomography (PET) is another suitable technique for detecting radiation in a subject. Furthermore, U.S. Pat. No. 5,429,133 describes a laparoscopic probe for detecting radiation concentrated in solid tissue tumors. Miniature and flexible radiation detectors intended for medical use are produced by Intra-Medical LLC, Santa Monica, Calif. Magnetic Resonance Imaging (MRI) or any other imaging technique known to one of skill in the art is also suitable for detecting the radioactive emissions of radionuclides. Regardless of the method or device used, such detection is aimed at determining where the RIC is concentrated in a subject, with such concentration being an indicator of the location of a tumor or tumor cells.

In still yet another aspect, the present invention provides a kit for radiotherapy comprising:
(a) a first container holding a radioimmunoconjugate having a radiopharmaceutical attached via a linking group to a targeting antibody or antibody fragment, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein the linking group contains a cleavage site recognized by a co-administered protease;
(b) a second container holding the co-administered protease; and
(c) directions for use of the radioimmunoconjugate (RIC) and the co-administered protease in radiotherapy.

In one embodiment, the radiopharmaceutical is a radionuclide bound to a chelating agent. In another embodiment, the linking group is radiolabeled with a radionuclide. Suitable radionuclides include, but are not limited to, ⁴⁷Sc, ⁶⁴Cu, ⁶⁷Cu, ⁸⁹Sr, ⁸⁶Y, 87Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. In yet another embodiment, the linking group is radiolabeled with ¹⁸F, ¹²⁴I, ¹²⁵I, ¹³¹I, or mixtures thereof. Suitable chelating agents include, but are not limited to, DOTA, BAD, TETA, DTPA, EDTA, NTA, HDTA, their phosphonate analogs, and mixtures thereof. In a preferred embodiment, the radiopharmaceutical is ⁹⁰Y bound to DOTA. The targeting antibody or antibody fragment that is attached to the radiopharmaceutical is preferably a monoclonal antibody. In a further embodiment, the monoclonal antibody is selected from the group consisting of anti-ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibodies. The linking group that attaches the radiopharmaceutical to the targeting antibody is preferably a peptide comprising from three to twenty amino acids. In a preferred embodiment, the linking group comprises CZ-amino acids in both the D-configuration and L-configuration. In a particularly preferred embodiment, the linking group comprises a heptapeptide having the sequence -rqYKYkf- (SEQ ID NO:1) or a conservatively modified variant thereof.

In another embodiment, the protease that recognizes a cleavage site on the linking group is selected from the group consisting of endopeptidases, serine proteases, metalloproteases, exopeptidases, carboxypeptidases, aminopeptidases, and the like. More particularly, proteases such as tissue-type plasminogen activator (t-PA), cathepsin B, cathepsin D, trypsin, chymotrypsin, and pepsin are suitable for use in the present invention. Preferably, the protease is t-PA or a modified form thereof. In preferred embodiments, the protease is a modified form of t-PA, such as Activase® or TNKase®. In a particularly preferred embodiment, the protease is TNKase®. The term “co-adminstered” refers to a protocol wherein the protease is either administered at the same time as the RIC or the protease and RIC are administered sequentially in a predetermined order. In a preferred embodiment, the protease is administered to a subject after the RIC. The time of protease administration following RIC administration, or “intervention time,” is influenced by a number of factors, such as blood clearance rates, tumor uptake and clearance rates, and radionuclide decay rates. Preferably, the intervention time is between 2 and 24 hours. More preferably, the intervention time is at about 6 hours.

In a further aspect, the present invention provides a kit for radioimaging comprising:
(a) a first container holding a radioimmunoconjugate having a radiopharmaceutical attached via a linking group to a targeting antibody or antibody fragment, the linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein the linking group contains a cleavage site recognized by a co-administered protease;
(b) a second container holding the co-administered protease; and
(c) directions for use of the radioimmunoconjugate and the co-administered protease in radioimaging.

In one embodiment, the radiopharmaceutical is a radionuclide bound to a chelating agent. In another embodiment, the linking group is radiolabeled with a radionuclide. Suitable radionuclides include, but are not limited to, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ^{99m}Tc, ²⁰¹Tl, and mixtures thereof. In yet another embodiment, the linking group is radiolabeled with ¹⁸F, ¹³¹I, or mixtures thereof. Suitable chelating agents include, but are not limited to, DOTA, BAD, TETA, DTPA, EDTA, NTA, HDTA, their phosphonate analogs, and mixtures thereof. In a preferred embodiment, the radiopharmaceutical is ⁶⁴Cu or ¹¹¹In bound to DOTA. The targeting antibody or antibody fragment that is attached to the radiopharmaceutical is preferably a monoclonal antibody. In a further embodiment, the monoclonal antibody is selected from the group consisting of anti-ChL6, Lym-1, CD1lb, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibodies. The linking group that attaches the radiopharmaceutical to the targeting antibody is preferably a peptide comprising from three to twenty amino acids. In a preferred embodiment, the linking group comprises α-amino acids in both the D-configuration and L-configuration. In a particularly preferred embodiment, the linking group comprises a heptapeptide having the sequence -rqYKYkf- (SEQ ID NO:1) or a conservatively modified variant thereof.

In another embodiment, the protease that recognizes a cleavage site on the linking group is selected from the group consisting of endopeptidases, serine proteases, metalloproteases, exopeptidases, carboxypeptidases, aminopeptidases, and the like. More particularly, proteases such as tissue-type plasminogen activator (t-PA), cathepsin B, cathepsin D, trypsin, chymotrypsin, and pepsin are suitable for use in the present invention. Preferably, the protease is t-PA or a modified form thereof. In preferred embodiments, the protease is a modified form of t-PA, such as Activase® or TNKase®. In a particularly preferred embodiment, the protease is TNKase®. The term “co-administered” refers to a protocol wherein the protease is either administered at the same time as the RIC or the protease and RIC are administered sequentially in a predetermined order. In a preferred embodiment, the protease is administered to a subject after the RIC. The time of protease administration following RIC administration, or “intervention time,” is influenced by a number of factors, such as blood clearance rates, tumor uptake and clearance rates, and radionuclide decay rates. Preferably, the intervention time is between 2 and 24 hours. More preferably, the intervention time is at about 6 hours.

IV. Compositions: Radioimmunoconjugates (RICs)

A radioimmunoconjugate (RIC) of the present invention is comprised of three separate elements: a radiopharmaceutical, an antibody, and a peptide linking group covalently attaching the radiopharmaceutical to the antibody. A protease is employed to selectively cleave a site in the peptide linking group. Each of these elements will be described in greater detail below.

A. Radiopharmaceuticals

In preferred embodiments of the present invention, the biological agent (e.g., a therapeutic, imaging, or anticancer agent) is a radiopharmaceutical. Suitable radiopharmaceuticals include, but are not limited to, radionuclides, radionuclides directly coupled to an antibody, radionuclides directly coupled to a linking group, and radionuclides bound to a chelating agent (“radio-metal chelate”). Preferably, the radiopharmaceutical of the present invention comprises a radionuclide bound to a chelating agent.

Radionuclides suitable for use in the present invention include, but are not limited to, fluorine 18 (¹⁸F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y), rhodium 105 (¹⁰⁵Rh), silver 111 (¹¹¹Ag), indium 111 (¹¹¹In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹Tl), astatine 211 (²¹¹At), and bismuth 212 (²¹²Bi). As used herein, the “m” in ^{117m}Sn and ^{99m}Tc stands for meta state. Additionally, naturally occurring radioactive elements such as uranium, radium, and thorium, which typically represent mixtures of radioisotopes, are suitable examples of radionuclides. ⁶⁷Cu, ¹³¹I, ¹⁷⁷Lu, and ¹⁸⁶Re are beta- and gamma-emitting radionuclides. ²¹²Bi is an alpha- beta-emitting radionuclide. ²¹¹At is an alpha-emitting radionuclides. ³²P, ⁴⁷Sc, ⁸⁹Sr, ⁹⁰Y, ¹⁰⁵Rh, ¹¹¹Ag, ^{117m}Sn, ¹⁴⁹Pm, ¹⁵³Sm, ¹⁶⁶ Ho, and ¹⁸⁸Re are examples of beta-emitting radionuclides. ⁶⁷Ga, ¹¹¹In, ^{99m}Tc, and ²⁰Tl are examples of gamma-emitting radionuclides. ⁵⁵Co, ⁶⁰Cu, ⁶Cu, ⁶²Cu, ⁶⁶Ga, ⁶⁸Ga, ⁸²Rb, ⁸⁶Y are examples of positron-emitting radionuclides. ⁶⁴Cu is a beta- and positron-emitting radionuclide.

⁹⁰Y is an attractive radionuclide for RIT because it provides greater tumor retention and more energetic beta emissions for killing cancerous cells when delivered to a tumor than ¹³¹I (Sharkey et al., Cancer Res., 48:3270-3275 (1988); Halpern et al., Cancer Res., 43:5347-5355 (1983); Pimm et al., Eur. J. Nucl. Med., 11:300-304 (1985)). ⁶⁴Cu or ¹¹¹In, when incorporated into an RIC, can be used to produce an image of the tumor, or the ⁶⁴Cu-RIC or ¹¹¹In-RIC can be mixed with a corresponding ⁹⁰Y-RIC to track the movement and localization of the ⁹⁰Y-RIC.

A chelating agent refers to a compound which binds to a metal ion, such as a radionuclide, with considerable affinity and stability. In certain preferred embodiments, the chelating agents of the present invention are bifunctional, having a metal ion chelating group at one end and a reactive functional group capable of binding to peptides, polypeptides, or proteins at the other end. Suitable bifunctional chelating agents include, but are not limited to, 1,4,7,10-tetraazacyclododecane-N,N′,N″,N′″-tetraacetic acid (DOTA), a bromoacetamidobenzyl derivative of DOTA (BAD), TETA, DTPA, ca-DTPA, SCNBzDTPA, and MxDTPA. Other chelating agents include EDTA, NTA, HDTA and their phosphonate analogs such as EDTP, HDTP, NTP.

In particularly preferred embodiments of the present invention, the radiopharmaceutical present in the RIC is either ⁹⁰Y bound to DOTA, ⁶⁴Cu or ¹¹¹In bound to DOTA, or mixtures thereof.

B. Antibodies

Antibodies suitable for use in the RICs of the present invention include, but are not limited to, monoclonal, polyclonal, and recombinant antibodies, as well as chimeric and humanized antibodies, and antibody fragments thereof. Preferably, the antibody is a monoclonal antibody or the antibody fragment is the Fab portion of a monoclonal antibody.

Monoclonal antibodies (MAbs) suitable for use in the present invention include, but are not limited to, ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibodies. The anti-adenocarcinoma chimeric L6 (ChL6) MAb reacts with an integral membrane glycoprotein found on some tumor cells. The anti-lymphoma (Lym-1) MAb recognizes a cell surface antigen on malignant B cells. The T101 anti-CD1b IgG1 MAb is suitable for use in the detection and/or treatment of cutaneous T-cell lymphomas. T3 is an example of an anti-CD3 IgG1MAb antibody. T1 is an example of an anti-CD5 IgG2a MAb. My4 is an example of an anti-CD14 IgG2b MAb. NKH1 is an example of an anti-CD56 IgG1MAb. The anti-CD20 MAb binds to the CD20 antigen found on normal and malignant B lymphomas. Rituximab, tositumomab, ibritumomab, B1, and 2B8 are examples of anti-CD20 MAbs. The anti-CD22 MAb antibody binds to the CD22 antigen. Epratuzumab is an example of a CD22 antibody. The anti-CD33 MAb binds to the CD33 antigen found on monocytes, activated T cells, granulocytes, myeloid progenitors, and mast cells. Gemtuzumab ozogamicin is a humanized anti-CD33 IgG4 MAb conjugated to calicheamicin, a complex oligosaccharide that makes double-stranded breaks in DNA. M195, an example of an anti-CD33 MAb, is a murine IgG2a MAb or humanized IgG1MAb that binds to the CD33 antigen. The anti-CD52 MAb binds to the CD52 antigen found on normal and malignant B and T lymphocytes and additional white blood cells. Alemtuzumab is an example of an anti-CD52 humanized IgG1MAb. The anti-HER2 MAb binds to HER2, a growth factor receptor found on some tumor cells. Trastuzumab is an example of an anti-HER2MAb. The anti-Tac MAb specific for the IL-2R α-chain is an example of an IL-2R antibody. The anti-EGF receptor MAb binds to the EGF receptor found on tumor cells. Cetuximab is an example of an anti-EGF receptor MAb. Anti-tenascin antibodies include, without limitation, BC-2 and 81C6 MAbs. Anti-TAG-72 antibodies include, without limitation, CC49 and B72.3 MAbs. Other antibodies suitable for use in the present invention include, but are not limited to, m170, BrE-3, hMN-14 (humanized anti-CEA Ab), 3F8, HMFG-1, HMFG-2, AUA1, H317, and H17E2.

C. Peptide Linking Groups

Phage display peptide library method: Prior to the advent of the present invention, a series of peptide substrates for t-PA was identified and characterized using a phage display peptide library method (Ding et al., Proc. Natl. Acad. Sci. USA, 92:7627-7631 (1995); Coombs et al., J. Biol. Chem., 273:4323-4328 (1998)). The consensus sequence for the P3, P2, P1, P1′, and P2′ substrate residues, wherein the site of cleavage is between P1 and P1′, was determined to be: X-(G>A)-R-X′-(A>G) wherein X′ was most often arginine, but could be other residues as well. The most active substrate had the sequence GGSG PFGR↓SA LVPEE, wherein “↓” denotes the cleavage site, in which PFGRSA was a hexapeptide sequence derived from the phage display peptide library screen and the flanking sequence was derived from the phage display vector. This peptide was hydrolyzed 5000-fold more efficiently by t-PA than a peptide of similar length derived from the native cleavage site of plasminogen. When this peptide sequence was inserted (i.e., cloned and expressed) between a 20 amino-terminal extension of T brucei ornithine decarboxylase, PFGRSA was efficiently cleaved by t-PA, with similar kinetics reported for activation cleavage of Lys-plasminogen by t-PA (Coombs et al., J. Biol. Chem., 271:4461-4467 (1996)). Interestingly, unlike plasminogen, which requires fibrin for efficient cleavage, cleavage of PFGRSA-ornithine decarboxylase occurred independent of fibrin. However, the hexapeptide PFGRSA is highly susceptible to cleavage by human plasma and tumor cell supernatants. As an “on-demand cleavable” linker (ODC-linker) must be stable in human plasma, i.e., resistant to cleavage or degradation from proteases found in plasma or tumor cells, this hexapeptide is clearly not suitable for use in the present invention.

“One-bead one-compound” combinatorial library method: Combinatorial library methods not only offer great potential for facilitating the drug discovery process, but also provide powerful tools for basic research in various disciplines (Lam, Anti-Cancer Drug Design, 12:145-167 (1997); Tiebes, In “Comb. Chem.” Ed. Weinheim, J. G. Wiley-VCH. pp. 1-34 (1999); Antonenko et al., Methods Princ. Med. Chem., 7:39-80 (2000); Lehn and Eliseev, Science, 291:2331-2332 (2001); Appell et al., Sep. Sci. Technol., 3:23-56 (2001)).

The “one-bead one-compound” (OBOC) combinatorial library method was first reported in 1991 (Lam et al., Nature, 354:82-84 (1991)). In essence, when a “split-mix” synthesis method (Lam et al., Nature, 354:82-84 (1991); Houghten et al., Nature, 354:84-86 (1991); Furka et al., Int. J. Peptide Protein Res., 37:487-493 (1991)) is used to generate a combinatorial library, each bead expresses only one chemical entity (Lam et al., Nature, 354:82-84 (1991); Lam et al., Chem. Rev., 97:411-448 (1997)). Random libraries of millions of beads can then be screened in parallel for a specific acceptor molecule (e.g., receptor, antibody, enzyme, virus, whole cell, etc.). Using an enzyme-linked colorimetric assay similar to that used in Western blotting, the OBOC combinatorial library method was successful in identifying ligands for an anti-β-endorphin antibody (Lam et al., Bioorg. Med. Chem. Lett., 3:419-424 (1993)), streptavidin (Lam et al., Pept.: Chem., Struct., Biol., Proc. Am. Pept. Symp. 13th, pp. 1005-1006 (1994)), avidin (Lam and Lebl, Immuno Methods, 1:11-15 (1992)), anti-insulin monoclonal antibody recognizing a discontinuous epitope (Lam et al., In “Peptides: Chem., Sturct., and Biol.” Ed. Hodges, pp. 1003-1004 (1994)), MHC-Class I molecules (Smith et al., Mol. Immunol., 31:1431-1437 (1994)), indigo carmine (a small organic dye) (Lam et al., Drug Dev. Res., 33:157-160 (1994)), and surface idiotype of B-cell lymphoma cell lines (Lam et al., Biomed. Pept, Prot., and Nuc. Acids, 1:205-210 (1995)). The positive beads were then physically isolated for structural determination by microsequencing using automatic Edman degradation (Lam et al., Nature, 354:82-84 (1991)).

The OBOC combinatorial library method can also be used for screening radiolabeled peptides. For example, substrate motifs for protein kinases were identified using peptides radiolabeled with [γ-³²P]-ATP. (Lam and Wu, Methods, 6:401-403 (1994); Wu et al., Biochem., 33:14825-14833 (1994); Lam et al., Intl. J. Prot. Pept. Res., 45:587-592 (1995); Lou et al., Bioorg. Med. Chem., 4:677-682 (1996)). Using these peptide substrates as templates, potent pseudo-substrate-based peptide inhibitors for p60^{c-src} protein tyrosine kinase were also developed (Alfaro-Lopez et al., J. Med. Chem., 41:2252-2260 (1998)). Since the OBOC combinatorial library method uses a parallel approach, each compound is spatially separated on individual beads, and multiple different peptide motifs can be identified (Wu et al., J. Comb. Chem. High-throughput screening (2002)). Recently, OBOC peptidomimetic libraries were used to identify peptidomimetic substrates for the development of c-src inhibitors (Kamath et al., In “Peptides: the wave of the future.” Proc. of Pept. Symp., Jun. 9-14, 2001).

Using the OBOC combinatorial library method, peptide substrates were screened for cleavage by proteases such as cathepsin B, cathepsin D, and the modified t-PA TNKase®. In particular, several random libraries were screened, including a pentapeptide library and a heptapeptide library, for substrates of TNKase®. The peptides were comprised mostly of α-amino acids having an L-configuration, although a single D-amino acid, as well as amino acid analogs and mimetics, were also used. These peptides also contained a quencher and a fluorescent dye flanking the sequences, such that cleavage of the peptide by TNKase® would release the quencher and allow the bead to fluoresce (Meldal et al., Proc. Natl. Acad. Sci., 91:2214-3318 (1994)). From an XXXX(f/F) pentapeptide library, wherein X is any L-amino acid, two distinct motifs were identified: GR(R/X)(G/X) and GD(N/D/G)(G/N/S), as shown in Tables 1A and 1B. From a heptapeptide library containing amino acid analogs and mimetics, six distinct t-PA peptide substrates were identified, as shown in Table 1C. Interestingly, non-naturally occurring amino acid residues such as norleucine (Nle), phenylglycine (Phg), and hydroxyproline (Hyp) were also present in some of the peptides.

TABLE 1
A.

| | P2 | P1 | P1′ | P2′ | P3′ | |
|---|---|---|---|---|---|---|
| Y(NO₂)- | X | X | X | X | f/F | -K(Abz) |
| | R | R | R | L | F | |
| | V | R | R | Q | F | |
| | V | R | R | G | F | |
| | G | R | T | N | F | |
| | G | R | R | T | F | |
| | G | R | N | G | F | |
| | A | R | A | G | F | |

B.

| X | X | X | X | f/F |
|---|---|---|---|---|
| G | D | N | G | F |
| G | D | D | S | F |
| G | D | D | G | F |
| G | D | N | N | F |
| G | D | G | G | F |
| E | D | G | G | F |

C.

| Y(NO₂)- | O | O | O | O | O | O | O | -K(Abz) |
|---|---|---|---|---|---|---|---|---|
| | I | Hyp | Nle | T | I | Hyp | P | |
| | X | Hyp | N | Hyp | Hyp | X | D | |
| | Hyp | P | A | Q | I | Nle | X | |
| | Nle | V | R | Phg | Hyp | P | E | |
| | W | G | N | N | D | D | D | |
| | D | D | D | L | E | E | X | |

Although several of these peptide substrates were indeed susceptible to cleavage by TNKase®, most of them, however, were proteolytically unstable in human plasma. As such, similar to the PFGRSA hexapeptide of Coombs et al., these peptides would not be effective ODC-linkers. Therefore, a new strategy is needed for the development of such linkers.

OBOC combinatorial library method using D- and L-amino acids: It is well known that there are many different proteases present in the blood circulation. As a result, in order for an RIC to be highly specific, potent, and therapeutically effective, its ODC-linker must be stable in plasma and resistant to proteases present therein. Although the P1 and P1′ sites are the major determining factors for proteolysis of the peptide by a specific enzyme, other flanking residues such as P2, P3, P2′, and P3′ also affect both the specificity and efficiency of cleavage. For example, in an all L-amino acid-containing peptide linker (e.g., a hexapeptide), while the exogenous protease selectively cleaves the P1-P1′ site, proteases in plasma may cleave peptide bonds outside of this site. Although restricting the linker to a dipeptide may solve this problem, the dip eptide linker may not contain sufficient interaction sites with the exogenous protease to generate high enough specificity and proteolytic efficiency.

To solve this problem, an octapeptide library with the middle two amino acids having an L-configuration and the remaining flanking residues having a D-configuration (i.e., an xxxXXxxx octapeptide, wherein x is a D-amino acid and X is an L-amino acid) was screened with both trypsin and TNKase®. As shown in Table 2, the seven substrates identified using trypsin were surprisingly cleaved between an L-Arginine (R) at position 5 and a D-amino acid at position 6 in the octopeptide sequence. Although cleavage at the carboxyl side of a basic residue such as arginine or lysine is characteristic for trypsin, the unexpected findings were: (i) the preference of trypsin for arginine, but not lysine; and (ii) the cleavage between an L-amino acid and a D-amino acid, rather than between two L-amino acids, is preferred. The latter finding has great implication in the development of ODC-linkers using TNKase®, as it suggests that highly specific substrates can be identified with this approach.

TABLE 2

| | | | P3 | P2 | P1 | P1′ | P2′ | P3′ | |
|---|---|---|---|---|---|---|---|---|---|
| K(Dnp)- | x | x | x | X | X | x | x | x | -k(Abz) |
| | r | g | n | E | R | y | r | g | |
| | y | t | v | Thi | R | s | v | q | |
| | q | y | m | A | R | l | l | a | |
| | p | q | s | M | R | v | n | q | |
| | r | e | r | Y | R | r | q | l | |
| | l | r | e | H | R | r | e | r | |
| | y | l | t | V | R | t | n | f | |

(lower case = D-amino acids; upper case = L-amino acids)

Although screening the octopeptide library with TNKase® did not produce any strong candidates, screening a heptapeptide library with the middle three amino acids having an L-configuration and the remaining flanking residues having a D-configuration (i.e., an xxXXXxx heptapeptide, wherein x is a D-amino acid and X is an L-amino acid) using TNKase® resulted in the identification of several substrates. Approximately 100,000 random peptide beads were first pre-screened by incubating with human plasma for three hours, and all the positive beads were isolated under a fluorescent microscope. TNKase® (200 μg) was then added to the dish and incubated for another hour. Positive beads were then isolated for sequence analysis, and the corresponding heptapeptide sequences are shown in Table 3.

TABLE 3

| | | | | P2 | P1 | P1′ | P2′ | P3′ | |
|---|---|---|---|---|---|---|---|---|---|
| Peptide # | Y(NO₂)- | x | x | X | X | X | x | x | -k(Abz) |
| 11 | | p | G | R | R | H | k | p | |
| 12 | | y | v | R | K | Q | w | k | |
| 13 | | p | d | A | R | K | f | n | |
| 14 | | p | k | G | K | K | G | e | |
| 15 | | l | r | N | K | K | G | G | |
| 16 | | k | q | Y | K | Y | k | l | |
| 17 | | f | l | H | K | S | r | i | |
| 18 | | f | r | Q | K | W | G | k | |
| 19 | | G | r | F | K | M | a | t | |
| 20 | | r | q | Y | K | Y | k | f | |

(lower case = D-amino acids; upper case = L-amino acids)

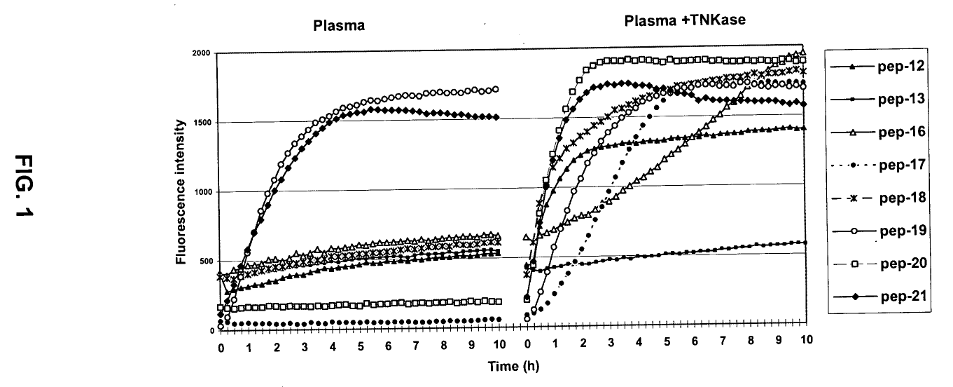

The peptides in Table 3 were re-synthesized in soluble form with nitrotyrosine (Y(NO₂) at the N-terminus and Abz at the ε-amino group of the lysine residue (k(Abz)) at the C-terminus. They were then incubated with human plasma alone or plasma plus TNKase® in a 96-well plate and the cleavage reaction was monitored over a period of time with a fluorescent plate reader. Each well contained 90 μM of peptides, 10 μl of plasma in PBS, ph 7.2, with or without 1 μg of TNKase®, in a final volume of 100 μl. Cleavage was measured as fluorescence intensity at different time intervals in the fluorescence plate reader at 360/440 nm excitation and emission range. These results are shown in FIG. 1.

Peptides #17 and #20 were determined to be the best candidates for ODC-linker development because both of these peptides are stable in human plasma and susceptible to TNKase® cleavage. This is especially true for peptide #20 (rqYKYkf), which is completely stable in plasma, but rapidly degraded by TNKaset®. In fact, within two hours of incubation with a clinically achievable dose of TNKase® (10 μg/ml), peptide #20 was completely degraded. By contrast, both peptide #19 and the PFGRSA peptide of Coombs et al., (1998) were highly susceptible to cleavage in plasma. This is not surprising as the PFGRSA peptide consists of all L-amino acids and therefore contains other susceptible sites besides the TNKase® cleavage site.

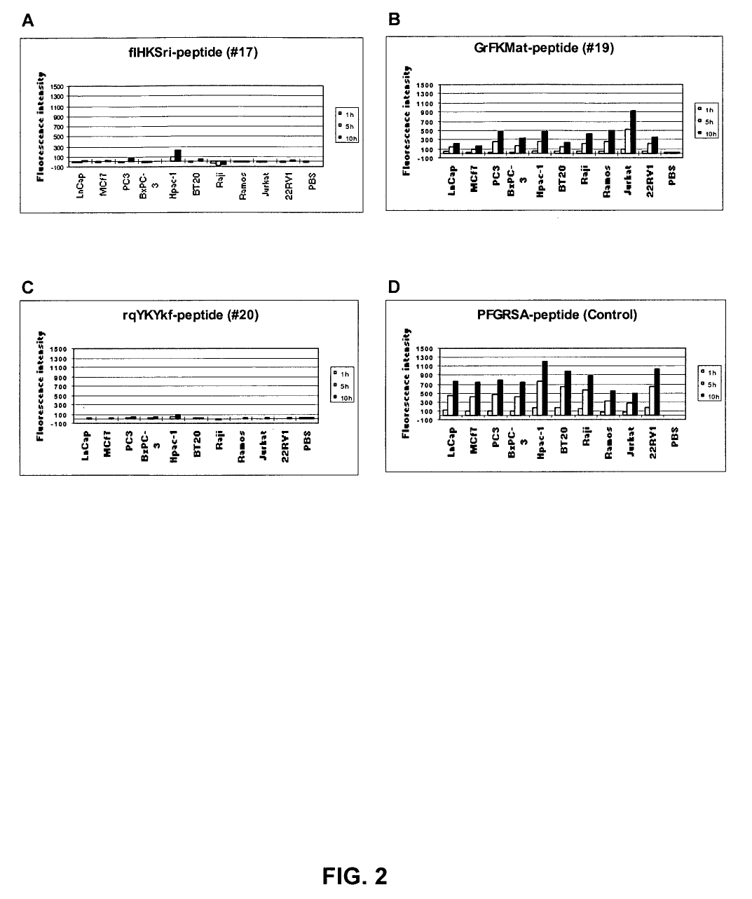

Further, to be an effective ODC-linker, a peptide must also be stable against proteases secreted by tumor cells. FIG. 2 illustrates the stability of peptides #17, #19, and #20, as well as the PFGRSA peptide, in supernatants collected from several tumor cell line cultures. Special care was taken to ensure that the tumor cells were growing in log phase and that the viability of these cells were in excess of 99%. Whereas peptide #19 and the PFGRSA peptide were highly susceptible to cleavage by all tumor cell culture supernatants, peptides #17 and #20 were highly resistant to proteolysis by tumor cell culture supernatants. Thus, peptides #17, #20, and conservatively modified variants thereof, are preferred peptides for use as ODC-linkers, as they possess the characteristics of rapid, specific cleavage by TNKase® but are stable in human plasma and tumor cell culture supernatants.

By using the OBOC combinatorial library method, novel peptide linkers that are susceptible to cleavage by TNKase® and yet stable in both plasma and tumor cell culture supernatants were successfully identified. These linkers are thus suitable for use as ODC-linkers in the RICs of the present invention.

D. Proteases

Suitable proteases for selectively cleaving the ODC-linker include, but are not limited to, endopeptidases (e.g., serine proteases and metalloproteases) and exopeptidases (e.g., carboxypeptidases and aminopeptidases). In particular, proteases such as cathepsin B, cathepsin D, trypsin, chymotrypsin, and pepsin are suitable for use in the present invention. Preferably, the protease is tissue-type plasminogen activator (t-PA) or a modified form thereof.

t-PA is a clot-dissolving serine protease produced naturally by cells in the walls of blood vessels and catalyzes the conversion of plasminogen to plasmin. Modified forms of t-PA include Activase® and TNKase®, both currently produced by and commercially available from Genentech. Activase® is currently being used in the clinic as a thrombolytic agent for patients with acute myocardial infarction, pulmonary embolism, and acute ischemic stroke. It has also been used clinically for dissolving clots in catheters and hemodialysis shunts.

Activase® is a purified glycoprotein of 527 amino acids produced by recombinant DNA technology, and has a chymotrypsin-like serine protease activity that initiates the fibrinolytic cascade by cleaving a single bond (Arg560-Val561) in the circulating zymogen plasminogen. This peptide bond is the only known physiologic substrate for t-PA. The remarkable specificity is in part due to the formation of a ternary complex between t-PA, plasminogen, and fibrin. As a result, the Kₘ of t-PA for plasminogen is lowered by more than 400-fold (Madison, J. of Biol. Chem., 270:7558-7562 (1995)). When introduced into the systemic circulation at pharmacologic concentrations, Activase® binds to firbrin in a thrombus and converts the entrapped plasminogen to plasmin. This initiates local fibrinolysis with limited systemic proteolysis. In a controlled clinical trial, 8 of 73 patients (11%) who received Activase® (1.25 mg/kg body weight over 3 hours) experienced a decrease in fibrinogen to below 100 mg/dl. Activase® has an initial plasma half-life of 5 minutes. The initial volume of distribution approximates plasma volume, and clearance is mediated primarily by the liver. The plasma clearance of Activase® is 380-570 ml/min.

Recently, the FDA has approved the marketing of a modified form of t-PA that has a longer plasma half-life. This new drug, called TNKase® or Tenecteplase, is also produced by recombinant DNA technology using an established mammalian cell line (Chinese Hamster Ovary cells). The amino acid sequence of TNKase® is identical to Activase® except for a substitution of threonine 103 with asparagine, a substitution of asparagine 117 with glutamine, both within the kringle 1 domain, and a substitution within the protease domain of amino acids 296-299 with four alanines.

Similar to Activase®, TNKase® also binds to fibrin and converts plasminogen to plasmin. Further, its catalytic rate is also increased in the presence of fibrin. However, its initial plasma half-life and terminal phase half-life are significantly longer, at 20-24 minutes and 90-130 minutes, respectively, than that of Activase®. The initial volume of TNKase® distribution is weight-related and approximates plasma volume. This suggests the majority of the enzyme resides in the intravascular space. Liver metabolism is the major clearance mechanism for TNKase®. Because of the longer plasma half-life, TNKase® is administered as a bolus over 5 seconds in thrombolytic therapy. In contrast, Activase® has to be given as a continuous infusion over three hours. TNKase® is supplied as a sterile, lyophilized powder in a 50 mg vial under partial vacuum.

V. Methods

A. Protease Dosage and Administration

In preferred embodiments of the present invention, the peptide linker is selectively cleaved by TNKase®. To ensure patient safety, it is desirable that the concentration of TNKase® a required for efficient cleavage of these peptide linkers can be achieved by administrating a TNKase® dose at or below the clinically approved dose used in the treatment of myocardial infarction. Table 4 summarizes the pharmacokinetics and pharmacodynamics data for TNKase® in humans.

TABLE 4

Plasma half-life = 22 minutes (vs. 3.5 minutes for Activase ®)
Dosing in human = i.v. bolus of 30-50 mg (0.53 mg/kg
bodyweight) over 5-10 sec.
Biphasic disposition:
Initial disposition phase predominant; mean half-live 17-24
minutes; mean terminal half-live 65-132 minutes
Mean clearance = 105 ml/min.
Mean initial volume of distribution = 4.2-6.3 L,
approximating plasma volume
Volume of distribution at steady state = 6.1-9.9 L,
suggesting limited extravascular distribution or binding

The clinical dose approved by FDA for use in the treatment of myocardial infarction is 30-50 mg i.v. bolus (or 0.53 mg/kg bodyweight). This translates to an initial plasma level of approximately 5-10 μg/ml. In one embodiment, the peptide linkers of the present invention are efficiently cleaved at about half the approved dose of 0.53 mg/kg body weight i.v. bolus. In a preferred embodiment, the peptide linker contains the sequence -rqYKYkf- (peptide #20). As shown in FIG. 1, in vitro studies demonstrated that peptide #20 was completely degraded upon incubation with 10 μg/ml of TNKase® (a clinically achievable level) for two hours. As such, in another embodiment, the dose of TNKase® administered to a subject effective to increase the release of a therapeutic, imaging, or anticancer agent relative to the amount of release in the absence of TNKase® administration is between 1-20 μg/ml, preferably between 2.5-10 μg/ml, and most preferably 2.5, 5, or 10 μg/ml.

In yet another embodiment, peptide substrates with a low Kₘ and high Vₘₐₓ are used as linkers in the RICs of the present invention. Through optimization of peptides such as peptide #20 and the screening of additional OBOC combinatorial libraries, substrate efficiency can be substantially improved, giving rise to peptides with low Kₘ and high Vₘₐₓ values. The term “optimization” as used herein refers to methods for identifying the most efficient and most specific peptide substrates, and an “optimized” peptide substrate is one identified by such methods, as described below. As a result, optimization can translate to a lower dose of TNKase® required for peptide cleavage. In still yet another embodiment, optimization of a peptide substrate produces at least a 2-fold improvement in cleavage efficiency by a protease, preferably at least a 10-fold improvement. In a preferred embodiment, the dose of TNKase® administered to a subject effective to increase the release of a therapeutic, imaging, or anticancer agent relative to the amount of release in the absence of TNKase® administration, wherein the peptide linker is an optimized peptide substrate, is lower than the dose of TNKase® required for an unoptimized peptide substrate.

Administration ofthe protease may occur either at the same time as RIC administration, or the protease and RIC may be administered sequentially in a predetermined order. In a preferred embodiment, the protease is administered to a subject after the RIC is administered. The time of protease administration following RIC administration, or “intervention time,” is influenced by a number of factors, such as blood clearance rates, tumor uptake and clearance rates, and radionuclide decay rates. Preferably, the intervention time is between 2 and 24 hours. More preferably, the intervention time is at about 6 hours. The intervention time should be such that the protease increases the release of a therapeutic, imaging, or anticancer agent from the RIC relative to its release in the absence of the protease. In a preferred embodiment, TNKase® administration results in at least a 75% reduction of radionuclide concentration in the plasma of a subject relative to control subjects.

B. Methods for Optimization of Peptide Substrates

In one embodiment of the present invention, any peptide linker identified by screening OBOC combinatorial libraries is suitable for optimization. In a preferred embodiment, the peptide linker contains the sequence -rqYKYkf- (peptide #20).

The following methods are suitable for optimizing a peptide substrate:
(1) Standard structure-activity relationship (SAR) studies such as an alanine walk will be performed on the peptides of interest. Solution peptide analogues are prepared and evaluated for TNKase® cleavage. This approach enables the identification of positions in the peptide that are essential for activity.
(2) Secondary libraries are made with the essential positions identified in (1) fixed and the other positions randomized. These libraries are screened under higher stringency (e.g., 10-fold decrease in TNKase® concentration).
(3) Homolog libraries are prepared containing peptides in which each amino acid is biased to the one found at the corresponding position in the peptide of interest. Such libraries are screened for cleavage by TNKase®.

The secondary libraries are necessary for optimization because it is impractical to screen all of the peptides that are theoretically present in a library that has 7-8 positions randomized. These libraries have greater than 20⁷ possible molecules. However, by making focused secondary and homolog libraries, a large number of related peptides can be rapidly screened, preferably under conditions of higher stringency.

Structure activity relationship (SAR) studies. In one embodiment, amino acid residues in a peptide of interest that are essential for protease cleavage are identified by “alanine walk.” As such, substitution of each amino acid with alanine (D- or L-, depending on the position), is performed, one at a time. The substituted peptides are made in soluble form, purified by HPLC, and tested for TNKase® cleavage activity. This approach enables the determination of the critical residues in the peptide that are required for biological activity. Results from these studies enable the rational design of more focused secondary libraries for subsequent screening.

Synthesis of secondary and tertiary libraries. In another embodiment, secondary libraries are designed based upon the common motif/building blocks of the initial peptides from the primary library and SAR studies. The focused library is screened under a higher stringency such as diluting'TNKase® 10-fold or shortening the incubation time for cleavage. In addition, the concentration of peptide can also be reduced. As a result, peptides identified from the secondary library have lower Kₘ values for TNKase®. The screening step can be repeated for additional rounds using tertiary libraries, etc. until the peptide substrate is optimized for cleavage.

This strategy has been used to develop a peptide for a B-cell epitope that binds with 10-100-fold higher affinity than the initial peptide (Lam et al., Methods: A Companion to Methods in Enzymology, 9:482-493 (1996)) and more recently with a peptide that binds to α₄β₁ integrin of lymphoma cell lines (Lam et al., unpublished data). A similar optimization strategy has yielded peptide substrates for p60 c-src protein tyrosine kinase (Kₘ=25-50 μM) and pseudosubstrate inhibitors with IC₅₀ values as low as 0.1 μM (Lou et al., Cancer Res., 57:1877-1881 (1997); Alfaro-Lopez et al., J. Med. Chem., 41:2252-2260 (1998); Kamath et al., In “Peptides: the wave of the future.” Proc. of Pept. Symp., Jun. 9-14, 2001).

Synthesis of homologue libraries. In yet another embodiment, optimization of a peptide is performed through the use of homolog libraries in which building blocks found in the peptide at specific positions are incorporated into the library at that position. As a result, the library is biased toward analogues related to the peptide of interest (Lebl et al., Biopolymers (Peptide Science), 47:177-198 (1995)). Such libraries are constructed so that at each position in the sequence, there is a 50% likelihood that the amino acid from the peptide is present, while the other 50% is randomly represented by another amino acid. This approach generates libraries of peptides that are randomized around a common motif. These libraries are then screened for cleavage by TNKase® under higher stringency.

As such, the foregoing methods provide highly efficient and specific peptide substrates suitable for use as ODC-linkers.

C. Pharmacokinetic and Radiation Dosimetric Studies of ODC-linker RICs

In one embodiment, the RICs of the present invention are used for treating cancer, such that the dose of radiation to normal tissues and organs is decreased and the dose of radiation to the tumor is increased. As such, in one preferred embodiment, the RICs of the present invention reduce blood radioisotope level by about 90%, while maintaining tumor radiation dose so that the tumor body therapeutic index is increased by at least 70% over that observed without protease intervention. Other preferred embodiments will be apparent from pharmacokinetic and radiation dosimetric studies performed in mice and humans as described below.

1. Studies in Mice

Pharmacokinetics are obtained from female nude mice bearing an adenocarcinoma xenograft of a defined size, using DOTA-tagged ODC-linked RICs. Mice are injected with RICs containing ⁹⁰Y or ¹¹¹In. The pharmacokinetics of the RIC is assessed as previously described (DeNardo et al., J. Nucl. Med., 36:829-836 (1995); DeNardo et al., Anticancer Res., 18:4011-4018 (1998); Deshpande et al., J. Nucl. Med., 29:217-225 (1988)). Total body clearance is determined using a sodium iodide detector system. Blood clearance is monitored by taking periodic blood samples from the tail veins of the mice. At the time of sacrifice, the xenograft and normal tissues are removed, weighted, and counted in a gamma well counter to provide organ distribution data.

The fundamental design for the characterization of an ODC peptide-linked RIC consists of 3 time points, a fixed amount and time for TNKase® intervention, and the number of mice selected based on biostatistical considerations for data variability, phase of the study, etc. For each ODC peptide-linked RIC, data will be obtained at 1, 3, and 5 days, because experience has shown that these observation points are sufficient to characterize the pharmacokinetics/radiation dosimetry (cumulated activity) for RICs. Because TNKase® is an approved drug whose adverse event profile has been characterized, a size-adjusted dose for human versus mouse of TNKase® will be used unless in vitro degradation of ODC peptide-linked RIC or toxicity in mice clearly indicates that lower doses can or should be used.

These studies can be categorized into three phases designed to assure that: 1) the DOTA-tagged ODC peptide-linked RIC has pharmacokinetics similar to that shown for DOTA-tagged GGGF-linked RIC, a prototype linker in current clinical use; 2) the ODC peptide-linked RIC meets minimum requirements for TNKase® degradation in vivo, leading to blood clearance and reduction in radiation dose to normal tissues and organs; and 3) the radiation dose to tumors in mice given the ODC peptide-linked RIC followed by TNKase® is similar to that of DOTA-tagged GGGF-linked RIC and ODC peptide-linked RIC without TNKase® intervention.

In further embodiments, an RIC containing a particular ODC peptide linker is suitable for treating cancer in a subject if the following criteria are met. First, the ODC-linked RIC delivers an estimated radiation dose (cumulated activity) to a tumor that is similar to or better than the dose for the same RIC when linked by a GGGF peptide linker. In a preferred embodiment, the activity of the ODC-linked RIC is at least 75% of that observed for the corresponding GGGF-linked RIC. Second, the ODC-linked RIC leads to mean blood radioisotope levels being reduced by at least 75% when the pre-TNKase®-treated blood level is compared to the lowest blood level observed within 4 hours of administration of TNKase®. Third, the ODC-linked RIC reduces radiation dose (cumulated activity) to blood and body by at least 25%, compared to the corresponding GGGF-linked RIC or ODC-linked RIC without TNKase® intervention, when the ODC-linker RIC is administered followed by TNKase® intervention. Fourth, the ODC-linked RIC reduces the radiation dose to lungs, kidneys, and liver by at least 25%, compared to the corresponding GGGF-linked RIC or ODC-linked RIC without TNKase® intervention, when the ODC-linker RIC is administered followed by TNKase® intervention. Fifth, the radiation dose delivered to a tumor with the ODC-linked RIC followed by TNKase® intervention is similar to or better (e.g., at least 75%) than the dose for the same RIC when linked by a GGGF peptide or without TNKase® intervention.

As such, ODC-linked RICs studied in mice that meet the above criteria are suitable for pharmaceutical development and use for treating cancer and/or imaging tumors in humans.

The time for intervention has been determined using a “lumped parameter model.” (DeNardo et al., J. Nucl. Med., 34:1020-1027 (1993)). Because bivalent antibodies bind essentially irreversibly to their antigenic sites (Yuan et al., Cancer Res., 51:3119-3130 (1991); Kyriakos et al., Cancer Res., 52:835-842 (1992); DeNardo, et al., J. Nucl. Med., 34:1020-1027 (1993)), this model is valid at least from a practical point of view and as a first approximation. Time after administration of radiolabeled antibody for optimal intervention has been determined using modeling of observational data obtained from tracer studies for 2 GGGF-linked RICs in mice and 2 GGGF-linked RICs in patients (DeNardo et al., Clinical Cancer Res., “Preclinical evaluation of cathepsin-degradable peptide linkers for radioimmunoconjugates,” in press, (2003); DeNardo, et al., Clinical Cancer Res., “Enhanced therapeutic index of radioimmunotherapy in prostate cancer patients: Comparison of radiation dosimetry for DOTA-peptide versus 2IT-DOTA MAb linkage for RIT,” in press, (2003)) and from relevant literature. Modeling simulations of data have confirmed that blood clearance rates, tumor uptake and clearance rates, and radionuclide decay rate influence the optimal time for implementing intervention. In mice, although the preferred intervention time is at about 22 hours, the therapeutic indices were within 1% of their maximum values for times between 16 and 24 hours. In human subjects, due to the pharmacokinetic differences between humans and mice, the intervention time is between 2 and 24 hours. Preferably, the intervention time is at about 6 hours. However, regardless of the type of organism receiving an RIC and protease, the intervention time should be such that the protease increases the release of an unbound therapeutic, imaging, or anticancer agent from the RIC relative to its release in the absence of the protease. In a preferred embodiment, TNKase® administration results in at least a 75% reduction of radionuclide concentration in the plasma of a subject relative to control subjects. In another preferred embodiment, TNKase® administration results in at least a 75% reduction in blood and body concentrations of radioisotope, a 70% improvement in tumor to body therapeutic ratio, and a 35% improvement in tumor to bone marrow therapeutic ratio.

2. Studies in Human Subjects

ODC-linked RICs studied in mice that meet the criteria described above are suitable for pharmaceutical development and use for treating cancer and/or imaging tumors in human subjects. In preferred embodiments, the ODC-linked RICs for use in human clinical trials meet the following in vitro and in vivo requirements, summarized in Table 5 below.

TABLE 5

| | |
|---|---|
| In vitro testing: | Stable in human plasma |
| Criteria for selection | Stable in cancer cell line culture medium |
| | Rapid cleavage by t-PA in the absence of fibrin |
| In vivo testing: | without t-PA intervention, radiation dose to |
| | tumor at least 75% of GGGF-linked RIC |
| Criteria for selection | with t-PA intervention, mean blood radioisotope |
| | level ↓ by at least 75% within 4 hr after |
| | t-PA administration |
| | with t-PA intervention, ↓ radiation dose to |
| | blood and body by at least 25% that of |
| | GGGF-linked RIC or ODC-linker without t-PA |
| | intervention |
| | with t-PA intervention, ↓ radiation dose to |
| | kidney, liver, and lung by at least 25% that of |
| | GGGF-linked RIC or ODC-linker without t-PA |
| | intervention |
| | with t-PA intervention, tumor dose at least |
| | 75% of GGGF-linked RIC or ODC-linker |
| | without t-PA intervention |

As indicated above, t-PA was chosen as the first exogenous enzyme for the cleavage of ODC-linkers because the concept can easily be translated into clinical studies as clinical grade t-PAs are readily available. Since t-PA is a thrombolytic agent, bleeding is a concern when it is used in cancer patients. However, because the peptide substrate is efficiently and specifically cleaved by t-PA, the dose of t-PA necessary for cleavage of the peptide linker is lower than half the dose approved for use in myocardial infarction. In fact, using a clinically achievable level of TNKase® (10 μg/ml), peptide #20 was completely degraded within 2 hours. In contrast, this same peptide was completely stable in human plasma or culture supernatants from various tumor cell lines. In the unlikely event that TNKase® (longer acting form) causes excessive bleeding complications, Activase®, the shorter acting form with an initial plasma half-life of 5 minutes, may be used. A significant thrombolytic state in a patient once Activase® infusion has been terminated is not expected, but as a precaution, the patient's coagulation parameters could be checked and made sure that the patient is not in a thrombolytic state prior to giving t-PA.

Kidney toxicity in patients could be a potential problem for small and medium size RICs generated from the cleavage of the ODC-linker. However, the short peptide ODC-linkers of the present invention (e.g., heptapeptides of approximately 700 Daltons) should circumvent this problem. As such, the released radionuclide-chelate-cleaved linker will be small and likely to be rapidly excreted into the urine. Further, incorporating some acidic residues into the peptide linker or giving i.v. cationic amino acids at the time of t-PA administration (Behr et al., Euro. J of Nucl. Med., 25:201-212 (1998)), should lower uptake of the radiometal into the renal tubules, leading to rapid excretion into the urine. For example, peptide libraries with acidic residues (e.g., D-glutamic acids) at the carboxyl end are designed so that so that every linker identified from such library has two built-in negative charges.

### VI. EXAMPLES

The following examples are offered to illustrate, but not to limit, the claimed invention.

### Example 1

### Synthesis of OBOC Combinatorial Libraries

Random synthetic combinatorial libraries are synthesized by a “split synthesis approach” as previously described (Lam et al., Nature, 354:82-84 (1991); Houghton et al., Nature, 354:84-86 (1991); Furka et al., Int. J. Peptide Protein Res., 37:487-493 (1991)). Amino-PEGA beads with a substitution of 0.4 mmol/g and diameter of 100-150 μm are used as solid phase support (Novabiochem). Amino-PEGA resin (1 g) is swollen in 15 ml DMF overnight and washed with DMF twice. A solution of Fmoc-(L)-Lys(F-Boc)-OH (0.562 g, 1.2 mmol), HOBt (0.162 g, 1.2 mmol), and DIC (188 μl, 1.2 mmol) in 10 ml DMF is added to the resin. The mixture is agitated at room temperature for 1 h. Complete coupling is confirmed by ninhydrin test. The resin is then washed with DMF (10 ml×3), MeOH (10 ml×3), and DCM (10 ml×3). The ε-Boc group of Fmoc-(L)-Lys(ε-Boc)-PEGA is removed with 50% TFA/DCM (twice, 10 ml and 10 min. for each time). The resin is immediately washed with DCM (10 ml×2), 2.5% DIPEA/DCM (10 ml×3), DCM (10 ml×1), MeOH (10 ml×3), and DMF (10 ml×3). A solution of Boc-Abz-OH (0.475 g, 2.0 mmol), HOBt (0.270 g, 2.0 mmol) and DIC (313 μl, 2.0 mmol) in 10 ml DMF is added to the resin. The mixture is shaken at room temperature overnight. Complete coupling is confirmed by ninhydrin assay. The resin is washed with DMF (10 ml×3), MeOH (10 ml×3), and DCM (10 ml×3).

Standard solid phase peptide synthesis methods with Fmoc chemistry is used to synthesize the remaining peptide chain of the libraries (Atherton and Sheppard, “Current Protocols in Protein Science.” John Wiley & Sons, Inc. (1999)). In addition to natural amino acids, commercially available D-amino acids, amino acid analogs, amino acid mimetics, and synthetic amino acids are also used as building blocks. The resin is divided into numbers of aliquots and each reacted with a single Fmoc-amino acid. Coupling is initiated by the addition of a three-fold molar excess of benzotriazolyl-N-oxy-tris(dimethylamino)-phosphonium hexafluoro-phosphate (BOP), 1-hydroxybenzotriazole (HOBt,) and diisoproylethylamine (DIEA). The coupling reaction is driven to completion with a three-fold molar excess of Fmoc amino acids and monitored by the standard ninhydrin test. Subsequently, the resin aliquots are washed, mixed thoroughly, washed, deprotected by 20% piperidine, washed, and divided into aliquots again for the next cycle of coupling. After the required number of cycles is completed, the side-chain protecting groups are removed by Mixture K (82.5% trifluoroacetic acids, 5% water, 5% p-cresol, 2.5% ethanedithiol). The resin beads are then washed and stored in CH₃OH at 4° C.

Random peptide-bead libraries are constructed such that the random sequences will be flanked by a quencher (nitro-tyrosine) at the amino terminus and a fluorescent dye (amino-benzoyl group) at the carboxyl terminus. Upon cleavage in the peptide, the quencher is released, resulting in blue fluorescent beads. To increase chemical diversity, D-amino acids, amino acid analogs, amino acid mimetics, and synthetic amino acids are used in addition to L-amino acids in the construction of linear or branched pentapeptide, hexapeptide, heptapeptide, or octapeptide libraries. For example, if 40 different amino acids are possible for each position in a hexapeptide sequence, such a library will contain approximately 4×10⁹ peptides. Preferably, peptides synthesized comprise both L- and D-amino acids. Such peptides, while being selective for an exogenous protease, will be stable to proteases in plasma and tumor cell culture supernatants.

### Example 2

### Screening OBOC Combinatorial Libraries for Protease Substrates

Peptide libraries are prescreened with human plasma and tumor cell culture supernatants so that all peptides which are susceptible to cleavage by proteases present in the plasma or at the tumor site are eliminated prior to screening with a protease. Preferably, the protease is a modified form of t-PA such as Activase® and TNKase®. About 1 ml of beads (equivalent to approximately 400,000 beads) are used in each screening experiment. The peptide bead library is first washed 5×with phosphate buffered saline (PBS), pH 7.2, in a small polypropylene column (2 ml). 200 μl of freshly thawed human plasma and a mixture of tumor cell line culture supernatants are then added and incubated at 37° C. for 1.5 hr. After washing with PBS, the beads are transferred to small Petri dishes and inspected under an inverted fluorescent microscope (OLYMPUS 1X70) with a U-M620 (330-385 nm) filter. All of the blue fluorescent beads are carefully removed from the library with a hand-held micropipette and discarded. The remaining beads in the dish should be dark. This prescreened library is now ready for protease assays.

Prescreened bead libraries are washed 5×with PBS in a 2 ml column. To the column (bottom capped), 200 μl of freshly thawed TNKase® and 200 μl PBS are added, the top capped, and the mixture incubated at 37° C. with gentle shaking. After the first 1.5 hr of treatment, the resin beds are washed 5×with PBS and 5×with water. The beads are then transferred to several small Petri dishes and inspected under an inverted microscope. The strong blue fluorescent beads are removed for microsequencing with an ABI protein sequencer as previously described (Liu and Lam, Anal. Biochem., 295:9-16 (2001)).

Alternatively, the fluorescent beads can be analyzed, sorted, and identified using a fluorescent activated bead sorter, such as the COPAS™ System (Union Biometrica, Inc), which is specifically designed to sort bigger objects like the 100-250 μm PEGA-beads used in the library screen. The fluorescent beads from the prescreening step are initially selected with a bead sorter and discarded. The remaining non-fluorescent beads are then screened with TNKase®, and the resulting fluorescent beads selected with the bead sorter. The final positive beads will be inspected under a fluorescent microscope for confirmation of fluorescent labeling prior to microsequencing.

The following screen for TNKase® substrates was performed according to the above-described method using OBOC combinatorial libraries. In particular, several million beads were screened to identify TNKase®-specific peptide substrates that are: (a) stable in plasma and other body fluids; (b) not digested by plasma proteases and tissue proteases, especially at the tumor site; and (c) highly susceptible for external protease. The OBOC combinatorial libraries used for screening were: (1) Y(NO₂)-xxXxx-K(Abz); (2) Y(NO₂)-xxxXXxxx-K(Abz); and (3) Y(NO₂)-xxXXXxx-K(Abz); wherein x is a D-amino acid and X is an L-amino acid.

Peptides susceptible to plasma and tissue proteases were removed by incubating beads for at least 12 hours with non-specific proteases such as plasmin (5 mU/mL), trypsin (0.5%), and matrix metalloprotease-2 (MMP-2) (2 mU/ml). Beads containing peptides susceptible to enzymatic digestion fluoresced and were removed using the COPAS™ System. The pre-cleared bead library was then used to screen for TNKase®-specific peptide substrates. Beads were incubated with a clinical dosage of TNKase® (10 μg/mL) for 30 minutes and fluorescent beads were collected and sequenced using a microsequencer. The peptide sequences identified from the OBOC combinatorial libraries are shown in Table 6.

TABLE 6

| | |
|---|---|
| xxXxx Library | xxXXXxx Library |

| | | | |
|---|---|---|---|
| pGFfe | ykRiy | myGRGrr | kyWRYyk |
| pGSrr | ykRki | nmGRWmk | |
| pGPhs | hGKpG | ymKRFka | |
| pGPhh | yGKsG | tkARWka | |
| akTpG | | rrRRGmw | |
| akQpG | | rGRRKGk | |

(lower case = D-amino acids; upper case = L-amino acids)

Some of the identified peptide sequences are unique and do not have basic amino acids such as arginine (R) or lysine (K) at the cleavage site. TNKase® was also observed to cleave peptide bonds between D-proline (p) and Glycine(G).

### Example 3

### Evaluation of Susceptibility of Peptide Linkers to Proteolysis: Specificity, Kₘ, and Vₘₐₓ.

Positive peptide substrates are resynthesized to confirm their specificities by determining the cleavage of these peptides by tumor cell supernatants, human plasma, purified tPA (Activase®, TNKase®), and urokinase using a solution phase assay. Urokinase is known to be present in some tumors and could potentially cleave the peptides. Peptide substrates used in these assays are constructed similar to the design of the peptides on the beads. In particular, the N-terminus of the peptide is capped by nitro-tyrosine and the carboxyl-terminal lysine contains an aminobenzoyl (Abz) moiety at its ε-amine. In the intact, uncleaved peptide, the aminobenzoyl-group (Abz) is quenched by Tyr(NO₂). However, after the peptide linker is cleaved, Abz is no longer quenched and fluoresces. The fluorescent intensity is then quantitated with a fluorescent plate reader. The assay is performed in a 96-well plate such that many peptides are evaluated concurrently with a fluorescent plate reader. Peptides that are stable in human plasma, urokinase, and in cancer cell line culture medium, but highly susceptible to cleavage by Activase® and TNKase® will be selected. Detailed enzyme kinetics studies are performed with various concentrations of peptide substrates and Activase® and/or TNKase®. Both Kₘ and Vₘₐₓ values for each peptide substrate are obtained. Peptides with low Kₘ and high Vₘₐₓ are selected for further development.

### Example 4

### Preparation of DOTA-Peptide-Antibody Conjugate

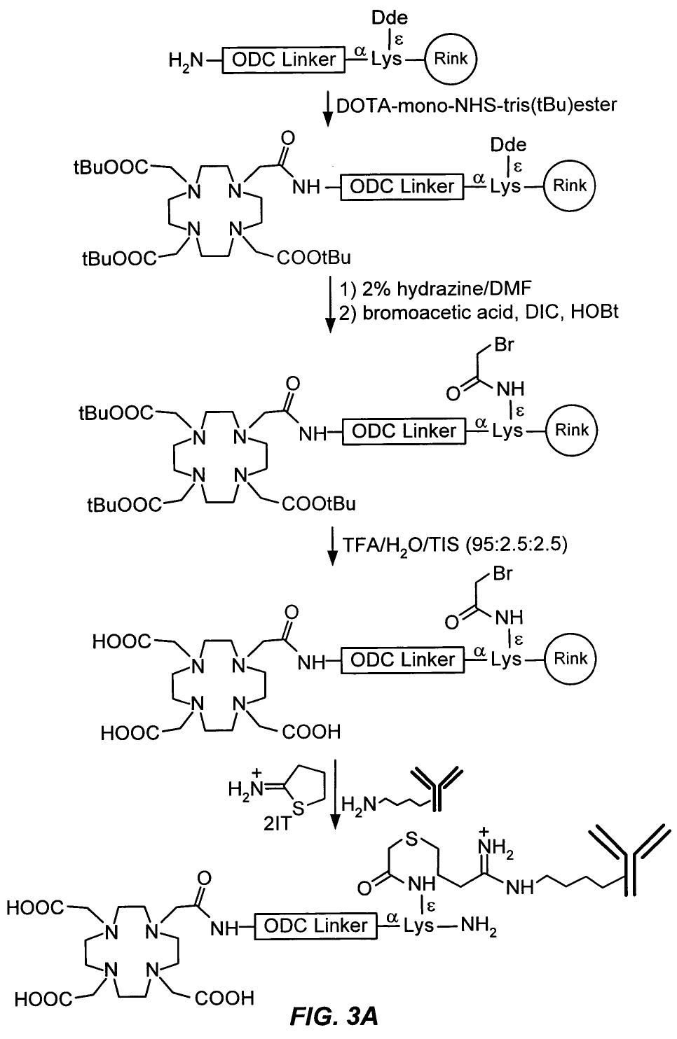

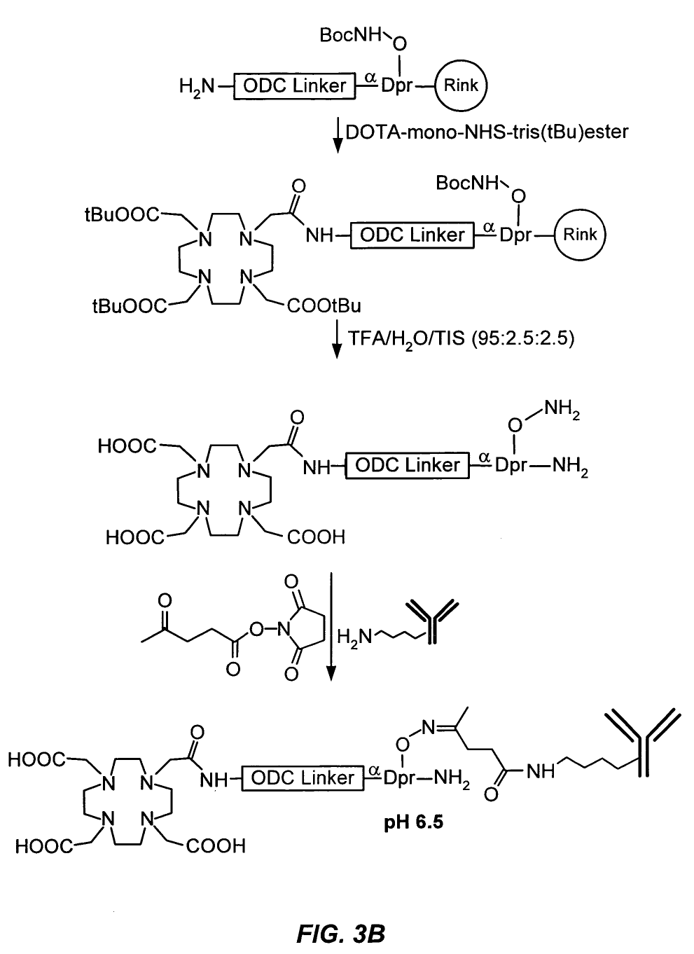

Many of the peptide linkers identified thus far contain one or more lysine residues (D- or L- amino acids), which are problematic for standard conjugation methods. Therefore, an orthogonal ligation approach was developed that can proceed efficiently and specifically in the presence of free amino groups on the peptide linker. Two such synthetic approaches have been developed, and are shown in FIG. 3. In both approaches, activated DOTA-peptide linkers are constructed on a solid support. After deprotection and cleavage off the resin, the activated linkers are purified using HPLC under metal-free conditions.

FIG. 3A illustrates a scheme wherein derivatizing the antibody molecule with 2-iminothiolane (Traut's reagent) allows the resulting free sulfhydryl groups on the antibody to react directly with the bromo-acetyl group of the peptide linker. After Fmoc deprotection at the N-terminus, the Rink resin bearing an ODC-linker is incubated with 1.2 equivalents (equiv.) of DOTA-mono-NHS-tris(tBu)ester (Macrocyclics, Dallas, Tex.) and 2.5 equiv. of DIEA in DMF until the ninhydrin test is negative. The supernatant is removed, and the resin is washed with DMF, methanol, and DMF again. The resin is then shaken with a 2% hydrazine solution in DMF for 2 minutes at room temperature. The supernatant is removed, and the process is repeated. After the resin is washed with DMF, DCM, methanol, and DMF again, a solution of 5 equiv. of bromoacetic acid, 5 equiv. of DIC, and 5 equiv. of HOBt in DMF is added. The resulting mixture is agitated until the ninhydrin test is negative. The supernatant is removed. The resin is washed with DMF, DCM, methanol, and DCM again, and dried under vacuum. To the dried resin, a mixture of TFA/H₂O/TIS (v/v/v 95:2.5:2.5) is added at an ice-bath temperature. The resulting mixture is slowly warmed to room temperature and allowed to mix for 2 h. The supernatant is separated and the resin is washed with methanol. The combined supernatant is concentrated to a small volume under a stream of nitrogen and then diluted with ethyl ether. The precipitate is separated, washed with ethyl ether, and purified by HPLC. The obtained DOTA-linker is subsequently conjugated to the antibody using standard methods (Michael et al., (1990)).

FIG. 3B shows a chemo-selective ligation strategy of first derivatizing a limited number of amino groups on the antibody molecule with N-succinimidyl levunilic acetate. The methyl-ketone group generated on the antibody then reacts site-specifically with the oxy-amino group of the linker to form an oxime bond, even in the presence of free amino and sulhydryl groups on the peptide linker. Fmoc-Dpr(BocAoa)—OH is attached to the Rink resin at the beginning of the synthesis, followed by Fmoc deprotection. The ODC linker is then constructed on the α-amino group of Dpr. After Fmoc deprotection at the N-terminus of the ODC-linker, the resin is incubated with 1.2 equiv. of DOTA-mono-NHS-tris(tBu)ester and 2.5 equiv. of DIEA in DMF until the ninhydrin test is negative. The supernatant is removed, and the resin is washed with DMF, DCM, methanol, and DCM again, and then dried under vacuum. To the dried resin, a mixture of TFA/H₂O/TIS (v/v/v 95:2.5:2.5) is added at an ice-bath temperature. The resulting mixture is slowly warmed to room temperature and allowed to mix for 2 h. The supernatant is separated and the resin is washed with methanol. The combined supernatant is concentrated to a small volume under a stream of nitrogen and then diluted with ethyl ether. The precipitate is separated, washed with ethyl ether, and purified by HPLC. The obtained DOTA-linker is subsequently conjugated to the antibody (Xu et al., 2003, in press).

### Example 5

### Characterization of an Antibody-ODC Linker-DOTA Conjugate

This example illustrates the synthesis, plasma stability, and TNKase® susceptibility of a ChL6-rqYKYkf-DOTA conjugate.

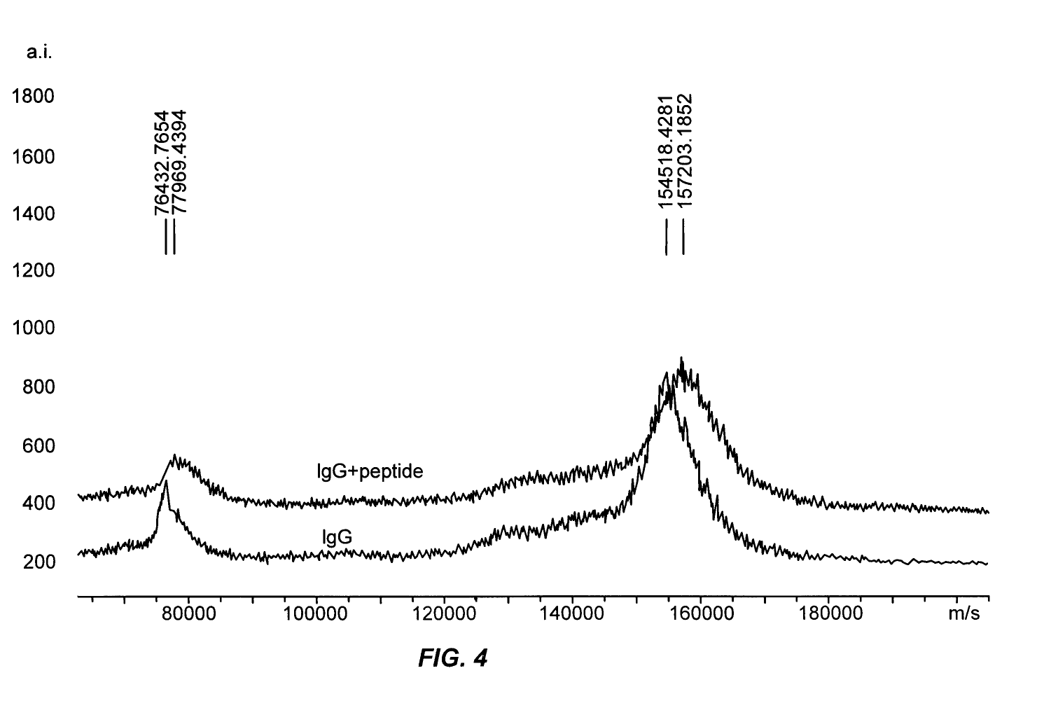

Conjugation of ODC linker to antibody: Conjugation of the -rqYKYkf- ODC linker to the breast cancer-specific antibody ChL6 was carried out using the ketone-oxime method described above (see, FIG. 3B). Briefly, a ketone-NHS linker was conjugated to the ChL6 antibody at a 1:30 molar ratio for 1 hour. Excess linker was removed by passing the conjugated antibody sample through a sephadex G-50 column. The amine group on the Dpr-Aoa-rqYKYkf-DOTA peptide was linked to the ketone group on the conjugated antibody linker to form a stable oxime bond at pH 6.5. The peptide conjugation to the antibody was confirmed by MALDI-TOF-mass spectrometry (FIG. 4). There was clear shift observed between the conjugated and unconjugated antibody. Based on the mass spectrometry analysis, about 1.4 peptides were conjugated per antibody molecule. The ChL6-rqYKYkf- DOTA conjugate was then radiolabeled with ¹¹¹In having a specific activity of 0.39 μCi/mg of conjugate.

Plasma stability assay: 300 μg of ¹¹¹In-labeled ChL6-rqYKYkf-DOTA conjugate was incubated in 1 mL of plasma for 14 days to check its stability, which was assessed by HPLC by checking counts at the antibody peak (155 kD). The ¹¹¹In-labeled ChL6-rqYKYkf- DOTA conjugate was stable for 7 days in plasma and the radiometal (¹¹¹In) remained complexed with the ChL6-rqYKYkf-DOTA conjugate without transferring to other plasma proteins.

Immunoreactivity assay: ¹¹¹In-labeled ChL6-rqYKYkf-DOTA conjugate was incubated with breast cancer cells (ChL6 reactive) and Raji cells (ChL6 non-reactive). Approximately one million cells were incubated for 1 hr and washed with PBS to remove any unbound conjugate. The cells were counted using a beta-counter. Table 7 shows that the conjugate selectively bound to specific breast cancer cells (HBT) and not to the control cells (Raji), indicating that conjugating the ODC linker to the antibody did not alter its antigen-binding site.

TABLE 7

| Amount of labeled | | | |
|---|---|---|---|
| conjugate used | | % of the radioactivity | % of the radioactivity |
| (0.39 μCi/mg) | | bound on HBT cells | bound on Raji cells |
| 10 | ng | 50.5 ± 0.3 | 6.6 ± 5.4 |
| 1 | ng | 49.0 ± 2.8 | 5.4 ± 4.6 |

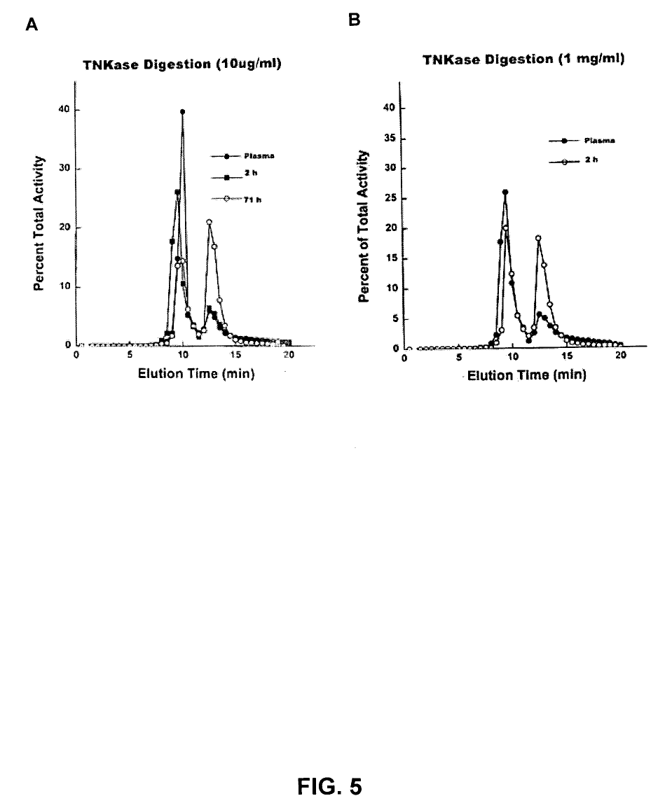

TNKase® susceptibility assay: ¹¹¹In-labeled ChL6-rqYKYkf-DOTA conjugate was incubated in plasma with and without TNKase®, which was used at the clinical dosage level of 10 μg/ml at different time points. Plasma samples were analyzed by HPLC and radioactivity and UV absorption (280 nm) were monitored. As shown in FIG. 5A, 10 μg/ml TNKase® digested˜30% of the ODC linker from the conjugate within 72 hours. The same level of digestion was observed at a higher TNKase® concentration (1 mg/ml) within 2 hours (FIG. 5B).

These results indicate that the ¹¹¹In-labeled ChL6-rqYKYkf-DOTA conjugate was stable to proteolysis by plasma, cleaved by TNKase® in vitro, and retained its tumor cell binding activity.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A conjugate comprising:
(i) a biological agent selected from the group consisting of a therapeutic agent, an imaging agent, and mixtures thereof;
(ii) a targeting moiety; and
(iii) a linking group covalently attaching said biological agent to said targeting moiety, said linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein said linking group is selectively cleaved by a protease.

2. A conjugate in accordance with claim 1, wherein said biological agent is a therapeutic agent.

3. A conjugate in accordance with claim 2, wherein said therapeutic agent is a radiopharmaceutical.

4. A conjugate in accordance with claim 3, wherein said radiopharmaceutical is a radionuclide bound to a chelating agent.

5. A conjugate in accordance with claim 4, wherein said radionuclide is selected from the group consisting of ⁴⁷Sc, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁸⁹Sr, ⁹⁰Y, ¹⁰⁵Rh, ¹¹¹Ag, ¹¹¹In, ^{117m}Sn, ^{99m}Tc. ¹⁴⁹Pm, ¹⁵³Sm, ¹⁶⁶Ho, ¹⁷⁷Lu, ¹⁸⁶Re, ¹⁸⁸Re, ²⁰¹Tl, ²¹¹At, ²¹²Bi, and mixtures thereof.

6. A conjugate in accordance with claim 4, wherein said chelating agent is DOTA.

7. A conjugate in accordance with claim 1, wherein said linking group is radiolabeled with a radionuclide.

8. A conjugate in accordance with claim 7, wherein said radionuclide is selected from the group consisting of ¹⁸F, ¹²⁴I, ¹²⁵I, ¹³¹I, and mixtures thereof.

9. A conjugate in accordance with claim 1, wherein said targeting moiety is an antibody or antibody fragment.

10. A conjugate in accordance with claim 9, wherein said antibody or antibody fragment is a monoclonal antibody.

11. A conjugate in accordance with claim 10, wherein said monoclonal antibody is selected from the group consisting of a ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibody.

12. A conjugate in accordance with claim 1, wherein said targeting moiety is selected from the group consisting of a small organic molecule, a peptide, a peptoid, a protein, and a polypeptide.

13. A conjugate in accordance with claim 1, wherein said linking group comprises from three to twenty amino acids.

14. A conjugate in accordance with claim 1, wherein said protease is selected from the group consisting of TNKase®, Activase®, and combinations thereof.

15. A conjugate in accordance with claim 1, wherein said biological agent is a radiopharmaceutical, said targeting moiety is a monoclonal antibody, and said linking group comprises a heptapeptide having the sequence -rqYKYkf- or a conservatively modified variant thereof, wherein said linking group is selectively cleaved by TNKase®.

16. A conjugate in accordance with claim 15, wherein said biological agent is ¹¹¹In bound to DOTA, said targeting moiety is an anti-ChL6 antibody, and said linking group comprises a heptapeptide having the sequence -rqYKYkf-, wherein said linking group is selectively cleaved by TNKase®.

17. A method for treating cancer in a subject in need thereof, said method comprising:
(a) administering to said subject a conjugate comprising an effective anticancer agent covalently attached to a targeting moiety by a cleavable linking group, said linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, wherein said linking group is stable in plasma and is selectively cleaved by a protease; and
(b) administering to said subject an amount of said protease effective to increase the release of unbound anticancer agent relative to the amount of release of said unbound anticancer agent in the absence of said protease.

18. A method in accordance with claim 17, wherein said anticancer agent is a radiopharmaceutical.

19. A method in accordance with claim 18, wherein said radiopharmaceutical is a radionuclide bound to a chelating agent.

20. A method in accordance with claim 19, wherein said radionuclide is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixture thereof.

21. A method in accordance with claim 19, wherein said chelating agent is DOTA.

22. A method in accordance with claim 17, wherein said linking group is radiolabeled with a radionuclide.

23. A method in accordance with claim 22, wherein said radionuclide is selected from the group consisting of ¹⁸F, ¹²⁴I, ¹²⁵I, ¹³¹I, and mixtures thereof.

24. A method in accordance with claim 17, wherein said targeting moiety is an antibody or antibody fragment.

25. A method in accordance with claim 24, wherein said antibody or antibody fragment is a monoclonal antibody.

26. A method in accordance with claim 25, wherein said monoclonal antibody is selected from the group consisting of a ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibody.

27. A method in accordance with claim 17, wherein said linking group comprises from three to twenty amino acids.

28. A method in accordance with claim 17, wherein said protease is selected from the group consisting of TNKase®, Activase®, and combinations thereof.

29. A method in accordance with claim 17, wherein said anticancer agent is a radiopharmaceutical, said targeting moiety is a monoclonal antibody, and said linking group comprises a heptapeptide having the sequence -rqYKYkf- or a conservatively modified variant thereof, wherein said linking group is selectively cleaved by TNKase®.

30. A method in accordance with claim 29, wherein said anticancer agent is ¹¹¹In bound to DOTA, said targeting moiety is an anti-ChL6 antibody, and said linking group comprises a heptapeptide having the sequence -rqYKYkf-, wherein said linking group is selectively cleaved by TNKase®.

31. A method for imaging a tumor, organ, or tissue, said method comprising:
(a) administering to a subject in need of such imaging, a conjugate comprising an imaging agent covalently attached via a linking group to a targeting moiety specific for said tumor, organ, or tissue, said linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein said linking group is selectively cleaved by a protease;
(b) detecting radiation from said imaging agent to determine where said conjugate is concentrated in said subject; and
(c) administering to said subject a protease that selectively cleaves said linking group to increase clearance of unbound imaging agent from said subject.

32. A method in accordance with claim 31, wherein said imaging agent is a radiopharmaceutical.

33. A method in accordance with claim 32, wherein said radiopharmaceutical is a radionuclide bound to a chelating agent.

34. A method in accordance with claim 33, wherein said radionuclide is selected from the group consisting of ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ^{99m}Tc, ²⁰¹Tl, and mixtures thereof.

35. A method in accordance with claim 33, wherein said chelating agent is DOTA.

36. A method in accordance with claim 31, wherein said linking group is radiolabeled with a radionuclide.

37. A method in accordance with claim 36, wherein said radionuclide is selected from the group consisting of ¹⁸F, ¹³¹I, and mixtures thereof.

38. A method in accordance with claim 31, wherein said targeting moiety is an antibody or antibody fragment.

39. A method in accordance with claim 38, wherein said antibody or antibody fragment is a monoclonal antibody.

40. A method in accordance with claim 39, wherein said monoclonal antibody is selected from the group consisting of a ChL6, Lym-1, CD1b, CD3, CD5, CD14, CD20, CD22, CD33, CD52, CD56, TAG-72, HER2/neu, interleukin-2 receptor (IL-2R), ferritin, neural cell adhesion molecule (NCAM), melanoma-associated antigen, ganglioside G_{D2}, EGF receptor, and tenascin antibody.

41. A method in accordance with claim 31, wherein said linking group comprises from three to twenty amino acids.

42. A method in accordance with claim 31, wherein said protease is selected from the group consisting of TNKase®, Activase®, and combinations thereof.

43. A method in accordance with claim 31, wherein said imaging agent is a radiopharmaceutical, said targeting moiety is a monoclonal antibody, and said linking group comprises a heptapeptide having the sequence -rqYKYkf- or a conservatively modified variant thereof, wherein said linking group is selectively cleaved by TNKase®.

44. A method in accordance with claim 43, wherein said imaging agent is ¹¹¹In bound to DOTA, said targeting moiety is an anti-ChL6 antibody, and said linking group comprises a heptapeptide having the sequence -rqYKYkf-, wherein said linking group is selectively cleaved by TNKase®.

45. A method in accordance with claim 31, further comprising co-administering a therapeutic agent, wherein said therapeutic agent is an anticancer agent covalently attached via said linking group to said targeting moiety.

46. A kit for radiotherapy comprising:
(a) a first container holding a radioimmunoconjugate having a radiopharmaccutical attached via a linking group to a targeting antibody or antibody fragment, said linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein said linking group contains a cleavage site recognized by a co-administered protease;
(b) a second container holding said co-administered protease; and
(c) directions for use of said radioimmunoconjugate and said co-administered protease in radiotherapy.

47. A kit for radioimaging comprising:
(a) a first container holding a radioimmunoconjugate having a radiopharmaceutical attached via a linking group to a targeting antibody or antibody fragment, said linking group comprising at least three amino acids, wherein at least two amino acids are selected from the group consisting of α-amino acids having a D-configuration, β-amino acids, γ-amino acids, N-substituted glycines, and combinations thereof, and at least one amino acid is an α-amino acid having an L-configuration, and wherein said linking group contains a cleavage site recognized by a co-administered protease;
(b) a second container holding said co-administered protease; and
(c) directions for use of said radioimmunoconjugate and said co-administered protease in radioimaging.


SEQUENCE LISTING (57 sequences)

SEQ ID NO: 1-10 - skipped (10 entries)

SEQ ID NO: 11 - protein, 7 aa, Artificial Sequence
  Description of Artificial Sequence: pentapeptide tissue-type plasminogen activator (t-PA) substrate containing flanking quencher and fluorescent dye
  MOD_RES (1): Xaa = nitrotyrosine (Y(NO-2))
  MOD_RES (7): Xaa = epsilon aminobenzoyl-lysine (K(Abz))
XRRRLFX

SEQ ID NO: 12 - protein, 7 aa, Artificial Sequence
  Description of Artificial Sequence: pentapeptide tissue-type plasminogen activator (t-PA) substrate containing flanking quencher and fluorescent dye
  MOD_RES (1): Xaa = nitrotyrosine (Y(NO-2))
  MOD_RES (7): Xaa = epsilon aminobenzoyl-lysine (K(Abz))
XVRRQFX

SEQ ID NO: 13 - protein, 7 aa, Artificial Sequence
  Description of Artificial Sequence: pentapeptide tissue-type plasminogen activator (t-PA) substrate containing flanking quencher and fluorescent dye
  MOD_RES (1): Xaa = nitrotyrosine (Y(NO-2))
  MOD_RES (7): Xaa = epsilon aminobenzoyl-lysine (K(Abz))
XVRRGFX

SEQ ID NO: 14 - protein, 7 aa, Artificial Sequence
  Description of Artificial Sequence: pentapeptide tissue-type plasminogen activator (t-PA) substrate containing flanking quencher and fluorescent dye
  MOD_RES (1): Xaa = nitrotyrosine (Y(NO-2))
  MOD_RES (7): Xaa = epsilon aminobenzoyl-lysine (K(Abz))
XGRTNFX

SEQ ID NO: 15 - protein, 7 aa, Artificial Sequence
  Description of Artificial Sequence: pentapeptide tissue-type plasminogen activator (t-PA) substrate containing flanking quencher and fluorescent dye
  MOD_RES (1): Xaa = nitrotyrosine (Y(NO-2))
  MOD_RES (7): Xaa = epsilon aminobenzoyl-lysine (K(Abz))
XGRRTFX

SEQ ID NO: 16 - protein, 7 aa, Artificial Sequence
  Description of Artificial Sequence: pentapeptide tissue-type plasminogen activator (t-PA) substrate containing flanking quencher and fluorescent dye
  MOD_RES (1): Xaa = nitrotyrosine (Y(NO-2))
  MOD_RES (7): Xaa = epsilon aminobenzoyl-lysine (K(Abz))
XGRNGFX

SEQ ID NO: 17 - protein, 7 aa, Artificial Sequence
  Description of Artificial Sequence: pentapeptide tissue-type plasminogen activator (t-PA) substrate containing flanking quencher and fluorescent dye
  MOD_RES (1): Xaa = nitrotyrosine (Y(NO-2))
  MOD_RES (7): Xaa = epsilon aminobenzoyl-lysine (K(Abz))
XARAGFX

SEQ ID NO: 18 - protein, 5 aa, Artificial Sequence
  Description of Artificial Sequence: pentapeptide tissue-type plasminogen activator (t-PA) substrate
GDNGF

SEQ ID NO: 19 - protein, 5 aa, Artificial Sequence
  Description of Artificial Sequence: pentapeptide tissue-type plasminogen activator (t-PA) substrate
GDDSF

SEQ ID NO: 20 - protein, 5 aa, Artificial Sequence
  Description of Artificial Sequence: pentapeptide tissue-type plasminogen activator (t-PA) substrate
GDDGF

SEQ ID NO: 21 - protein, 5 aa, Artificial Sequence
  Description of Artificial Sequence: pentapeptide tissue-type plasminogen activator (t-PA) substrate
GDNNF

SEQ ID NO: 22 - protein, 5 aa, Artificial Sequence
  Description of Artificial Sequence: pentapeptide tissue-type plasminogen activator (t-PA) substrate
GDGGF

SEQ ID NO: 23 - protein, 5 aa, Artificial Sequence
  Description of Artificial Sequence: pentapeptide tissue-type plasminogen activator (t-PA) substrate
EDGGF

SEQ ID NO: 24 - protein, 9 aa, Artificial Sequence
  Description of Artificial Sequence: heptapeptide tissue-type plasminogen activator (t-PA) substrate containing flanking quencher and fluorescent dye
  MOD_RES (1): Xaa = nitrotyrosine (Y(NO-2))
  MOD_RES (3): Xaa = hydroxyproline (Hyp)
  MOD_RES (4): Xaa = Nle
  MOD_RES (7): Xaa = hydroxyproline (Hyp)
  MOD_RES (9): Xaa = epsilon aminobenzoyl-lysine (K(Abz))
XIXXTIXPX

SEQ ID NO: 25 - skipped

SEQ ID NO: 26 - protein, 9 aa, Artificial Sequence
  Description of Artificial Sequence: heptapeptide tissue-type plasminogen activator (t-PA) substrate containing flanking quencher and fluorescent dye
  MOD_RES (1): Xaa = nitrotyrosine (Y(NO-2))
  MOD_RES (2): Xaa = hydroxyproline (Hyp)
  MOD_RES (7): Xaa = Nle
  MOD_RES (8): Xaa = any amino acid
  MOD_RES (9): Xaa = epsilon aminobenzoyl-lysine (K(Abz))
XXPAQIXXX

SEQ ID NO: 27 - protein, 9 aa, Artificial Sequence
  Description of Artificial Sequence: heptapeptide tissue-type plasminogen activator (t-PA) substrate containing flanking quencher and fluorescent dye
  MOD_RES (1): Xaa = nitrotyrosine (Y(NO-2))
  MOD_RES (5): Xaa = phenylglycine (Phg)
  MOD_RES (6): Xaa = hydroxyproline (Hyp)
  MOD_RES (9): Xaa = epsilon aminobenzoyl-lysine (K(Abz))
XXVRXXPEX

SEQ ID NO: 28 - protein, 9 aa, Artificial Sequence
  Description of Artificial Sequence: heptapeptide tissue-type plasminogen activator (t-PA) substrate containing flanking quencher and fluorescent dye
  MOD_RES (1): Xaa = nitrotyrosine (Y(NO-2))
  MOD_RES (9): Xaa = epsilon aminobenzoyl-lysine (K(Abz))
XWGNNDDDX

SEQ ID NO: 29 - protein, 9 aa, Artificial Sequence
  Description of Artificial Sequence: heptapeptide tissue-type plasminogen activator (t-PA) substrate containing flanking quencher and fluorescent dye
  MOD_RES (1): Xaa = nitrotyrosine (Y(NO-2))
  MOD_RES (8): Xaa = any amino acid
  MOD_RES (9): Xaa = epsilon aminobenzoyl-lysine (K(Abz))
XDDDLEEXX

SEQ ID NO: 30-53 - skipped (24 entries)

SEQ ID NO: 54 - protein, 6 aa, Artificial Sequence
  Description of Artificial Sequence: tissue-type plasminogen activator (t-PA) most active substrate hexapeptide derived from phage display peptide library screen
PFGRSA

SEQ ID NO: 55 - protein, 15 aa, Artificial Sequence
  Description of Artificial Sequence: tissue-type plasminogen activator (t-PA) most active substrate
GGSGPFGRSALVPEE

SEQ ID NO: 56 - protein, 4 aa, Artificial Sequence
  Description of Artificial Sequence: TNKase (Tenecteplase) modified tissue-type plasminogen activator (t-PA) substitution within protease domain of amino acids 296-299 with four alanines
AAAA

SEQ ID NO: 57 - protein, 4 aa, Artificial Sequence
  Description of Artificial Sequence: prototype peptide linker in radioimmunoconjugate (RIC) in current clinical use
GGGF